SEQUENCE LISTING (52 sequences)

SEQ ID NO: 1 - DNA, 20 bp, Artificial Sequence
  GAPDH-F Primer
ggcctccaaggagtaagacc

SEQ ID NO: 2 - DNA, 20 bp, Artificial Sequence
  GAPDH-R Primer
aggggtctacatggcaactg

SEQ ID NO: 3 - DNA, 20 bp, Artificial Sequence
  OCT3/4-F Primer
agtgagaggcaacctggaga

SEQ ID NO: 4 - DNA, 20 bp, Artificial Sequence
  OCT3/4-R Primer
acactcggaccacatccttc

SEQ ID NO: 5 - DNA, 20 bp, Artificial Sequence
  SOX2-F Primer
tggtacggtaggagctttgc

SEQ ID NO: 6 - DNA, 20 bp, Artificial Sequence
  SOX2-R Primer
tttttcgtcgcttggagact

SEQ ID NO: 7 - DNA, 20 bp, Artificial Sequence
  NANOG-F Primer
cagtctggacactggctgaa

SEQ ID NO: 8 - DNA, 20 bp, Artificial Sequence
  NANOG-R Primer
ctcgctgattaggctccaac

SEQ ID NO: 9 - DNA, 20 bp, Artificial Sequence
  SOX17-F Primer
ctgccacttgaacagtttgg

SEQ ID NO: 10 - DNA, 20 bp, Artificial Sequence
  SOX17-R Primer
cacacccaggacaacatttc

SEQ ID NO: 11 - DNA, 19 bp, Artificial Sequence
  FOXA2-F Primer
gagggctactcctccgtga

SEQ ID NO: 12 - DNA, 18 bp, Artificial Sequence
  FOXA2-R Primer
gcccacgtacgacgacat

SEQ ID NO: 13 - DNA, 20 bp, Artificial Sequence
  GATA4-F Primer
gctccttcaggcagtgagag

SEQ ID NO: 14 - DNA, 20 bp, Artificial Sequence
  GATA4-R Primer
gcccgtagtgagatgacagg

SEQ ID NO: 15 - DNA, 20 bp, Artificial Sequence
  DLK1-F Primer
atgctgcggaagaagaagaa

SEQ ID NO: 16 - DNA, 20 bp, Artificial Sequence
  DLK1-R Primer
tggtcatgtcgatcttctcg

SEQ ID NO: 17 - DNA, 20 bp, Artificial Sequence
  HNF1A-F Primer
gagcaaagaggcactgatcc

SEQ ID NO: 18 - DNA, 20 bp, Artificial Sequence
  HNF1A-R Primer
ctccagctctttgaggatgg

SEQ ID NO: 19 - DNA, 20 bp, Artificial Sequence
  HNF4A-F Primer
ctgtcccgacagatcacctc

SEQ ID NO: 20 - DNA, 20 bp, Artificial Sequence
  HNF4A-R Primer
gggatgtacttggcccactc

SEQ ID NO: 21 - DNA, 19 bp, Artificial Sequence
  KRT7-F Primer
agcaatgccctgagcttct

SEQ ID NO: 22 - DNA, 20 bp, Artificial Sequence
  KRT7-R Primer
gggtgggaatcttcttgtga

SEQ ID NO: 23 - DNA, 20 bp, Artificial Sequence
  KRT19-F Primer
agcaggtccgaggttactga

SEQ ID NO: 24 - DNA, 20 bp, Artificial Sequence
  KRT19-R Primer
gctcactatcagctcgcaca

SEQ ID NO: 25 - DNA, 20 bp, Artificial Sequence
  ALB-F Primer
aatgccctgtgcagaagact

SEQ ID NO: 26 - DNA, 20 bp, Artificial Sequence
  ALB-R Primer
ctgtgcagcatttggtgact

SEQ ID NO: 27 - DNA, 20 bp, Artificial Sequence
  AFP-F Primer
aaatgcgtttctcgttgctt

SEQ ID NO: 28 - DNA, 20 bp, Artificial Sequence
  AFP-R Primer
gccacaggccaatagtttgt

SEQ ID NO: 29 - DNA, 20 bp, Artificial Sequence
  AAT-F Primer
tctttgtgcctgttgctgtc

SEQ ID NO: 30 - DNA, 20 bp, Artificial Sequence
  AAT-R Primer
taccacaggggctattcagg

SEQ ID NO: 31 - DNA, 20 bp, Artificial Sequence
  TTR-F Primer
gcatgcagaggtggtattca

SEQ ID NO: 32 - DNA, 20 bp, Artificial Sequence
  TTR-R Primer
gccgtggtggaataggagta

SEQ ID NO: 33 - DNA, 20 bp, Artificial Sequence
  FABP1-F Primer
ctgcagagccaggaaaactt

SEQ ID NO: 34 - DNA, 20 bp, Artificial Sequence
  FABP1-R Primer
tctcccctgtcattgtctcc

SEQ ID NO: 35 - DNA, 18 bp, Artificial Sequence
  FGG-F Primer
ccaaacaggctggagacg

SEQ ID NO: 36 - DNA, 20 bp, Artificial Sequence
  FGG-R Primer
caacatggggtcttttgctc

SEQ ID NO: 37 - DNA, 20 bp, Artificial Sequence
  RBP4-F Primer
ggcagtacaggctgatcgtc

SEQ ID NO: 38 - DNA, 20 bp, Artificial Sequence
  RBP4-R Primer
ctgagggaagatggggagag

SEQ ID NO: 39 - DNA, 20 bp, Artificial Sequence
  TF-F Primer
ctcgggcaacttttgtttgt

SEQ ID NO: 40 - DNA, 20 bp, Artificial Sequence
  TF-R Primer
ggagtgatgaggtggagcat

SEQ ID NO: 41 - DNA, 20 bp, Artificial Sequence
  AHSG-F Primer
ggggaggatcagacacttca

SEQ ID NO: 42 - DNA, 20 bp, Artificial Sequence
  AHSG-R Primer
ataaccaccacccactctgc

SEQ ID NO: 43 - DNA, 20 bp, Artificial Sequence
  APOA4-F Primer
ggaacagctcaggcagaaac

SEQ ID NO: 44 - DNA, 20 bp, Artificial Sequence
  APOA4-R Primer
agctcagggagggagagagt

SEQ ID NO: 45 - DNA, 20 bp, Artificial Sequence
  HGF-F Primer
ggacgcagctacaagggaac

SEQ ID NO: 46 - DNA, 18 bp, Artificial Sequence
  HGF-R Primer
cccctcgaggatttcgac

SEQ ID NO: 47 - DNA, 21 bp, Artificial Sequence
  CYP1A2-F Primer
tgttcaagcacagcaagaagg

SEQ ID NO: 48 - DNA, 21 bp, Artificial Sequence
  CYP1A2-R Primer
tgctccaaagatgtcattgac

SEQ ID NO: 49 - DNA, 20 bp, Artificial Sequence
  CYP3A4-F Primer
gaaacacagatccccctgaa

SEQ ID NO: 50 - DNA, 20 bp, Artificial Sequence
  CYP3A4-R Primer
ctggtgttctcaggcacaga

SEQ ID NO: 51 - DNA, 20 bp, Artificial Sequence
  CYP2C9-F Primer
ggacagagacgacaagcaca

SEQ ID NO: 52 - DNA, 20 bp, Artificial Sequence
  CYP2C9-R Primer
catctgtgtagggcatgtgg

(19) United States
(12) Patent Application Publication
(10) Pub. No.: US 2013/0071931 A1
(43) Pub. Date: Mar. 21, 2013

(54) PROCESS FOR HEPATIC DIFFERENTIATION FROM INDUCED HEPATIC STEM CELLS, AND INDUCED HEPATIC PROGENITOR CELLS DIFFERENTIATED THEREBY

(71) Applicant: Tetsuya Ishikawa, Tokyo (JP)

(72) Inventor: Tetsuya Ishikawa, Tokyo (JP)

(73) Assignee: NATIONAL CANCER CENTER, Tokyo (JP)

(21) Appl. No.: 13/564,803

(22) Filed: Aug. 2, 2012

Related U.S. Application Data
(60) Provisional application No. 61/514,180, filed on Aug. 2, 2011.

(51) Int. Cl. C12N 5/071

(52) U.S. Cl. CPC C12N 5/067
USPC 435/377; 435/325

(57) ABSTRACT

A method for hepatic differentiation of a stem cell selected from among embryonic stem cells, induced pluripotent stem cells or induced hepatic stem cells is presented. More specifically, a stem cell selected from among embryonic stem cells, induced pluripotent stem cells or induced hepatic stem cells is cultured for 1 to 4 weeks in the presence of a TGF-β inhibitor, whereby the hepatic differentiation of the stem cell is realized.

### BACKGROUND OF THE INVENTION

1. Technical Field

The present invention relates to preparation of induced hepatic progenitor cells by culturing induced hepatic stem cells under specified culture conditions, as well as a method by which hepatocytes that have the similar features to a primary culture of hepatocytes and which can be used in non-clinical tests can be continuously prepared from induced hepatic stem cells or induced hepatic progenitor cells.

2. Background Art

In non-clinical tests currently conducted as part of the R&D efforts for new drugs, it is necessary to carry out pharmacological test using a number of animals and many types of animals in order to evaluate the safety, toxicity and other features of the drug under test and this is one of factors that leads to the soaring cost for the development of new drugs. What is more, the in vivo pharmacokinetics might differ on account of the species differences between human and other animals, making it difficult to perform sufficient evaluation of safety, toxicity and other features in animal tests, so it sometimes occurs that the candidate medicinal compound is not shown to have side effects before the clinical test stage is initiated.

Hence, there is a strong need to establish a system by which in vivo pharmacokinetics and the like of a candidate compound in humans can be predicted and evaluated at an early stage of the research and development processes, and efforts are now being made in order to construct an evaluation system that uses human hepatocytes. By using this evaluation system, candidate compounds for the drugs under development can be accurately limited to highly safe candidate drugs at an early stage of the development, so pharmaceutical companies have a particularly great demand for the system.

In conventional non-clinical tests using human cultured cells, primary cultured hepatocytes or existing cell lines from non-Japanese people have been employed. However, primary cultured hepatocytes have the problems of an overwhelming scarcity of donors and exceedingly great lot differences. Particularly notable problems are that primary cultured hepatocytes from Japanese people, which involve ethical issues and are regulated by law, are extremely difficult to obtain and cannot be supplied consistently.

Furthermore, enzymes that are associated with drug metabolizing systems and which are expressed in the liver tissue play an important role in drug metabolism. However, on account of the accompanying polymorphisms, the amount of their expression and their activity are affected by significant individual differences, so this problem of variation must be solved in a non-clinical test using primary cultures of hepatocytes can be performed successfully.

Given this situation, in order to eliminate the polymorphisms and individual differences, it is desirable that primary cultures of hepatocytes derived from a plurality of donors can be repeatedly used as representative cells in various types of tests. However, primary cultures of hepatocytes can hardly proliferate on a culture dish, so it is practically impossible to perform passage culture of the same hepatocyte and use it repeatedly in various tests.

In contrast, many of the existing established cell lines are those cells which have experienced karyotypic abnormality and there are not many enough cell lines to cover the polymorphisms and individual differences. Moreover, the existing established cell lines subjected to prolonged passage culture by conventional methods do not show the same drug metabolizing enzyme activity or inducing ability or transporter inducing ability as the primary culture of hepatocytes, so given this result, it is impossible to predict the safety, toxicity, metabolism, and other features in humans in clinical applications.

Under these circumstances, there have been desired cells that have the properties of hepatocytes and which can be supplied for an extended period.

For continuous supply of such useful hepatocytes, stem cells are required that allow livers to be supplied continuously. In the past, studies have been made to develop methods by which the differentiation of pluripotent stem cells such as embryonic stem cells or induced pluripotent stem cells into hepatocytes can be induced under various culture conditions. However, it is considered quite cumbersome and difficult to induce differentiation into hepatocytes by the methods studied so far.

In contrast, hepatic stem cells having the ability to differentiate into hepatocytes have been held promising as stem cells for the liver. The inventor of the present invention made intensive studies to prepare such hepatic stem cells and consequently demonstrated the possibility of preparing an induced hepatic stem cell that could be passage cultured ex vivo over an extended period, said stem cell expressing self-replicating genes like embryonic stem cells and induced pluripotent stem cells and also displaying properties characteristic of hepatocytes (PCT/JP 2011/000621; published as WO 2011/096223 on Aug. 11, 2011.) However, it cannot be considered optimal to use the thus prepared induced hepatic stem cell per se as a counterpart of primary cultures of hepatocytes.

### CITATION LIST

### Patent Literature

PATENT DOCUMENT 1: WO 2011/096223

### SUMMARY OF THE INVENTION

The present invention provides a method of differentiating an induced hepatic stem cell into an induced hepatic progenitor cell or a hepatocyte or a method of differentiating an induced hepatic progenitor cell into a hepatocyte, and an induced hepatic progenitor cell as a novel cell. More specifically, the present invention relates to a method of differentiating an induced hepatic stem cell into an induced hepatic progenitor cell or a hepatocyte, or differentiating an induced hepatic progenitor cell into a hepatocyte, by culturing the induced hepatic stem cell or induced hepatic progenitor cell for 1-4 weeks in the presence of a TGF-β inhibitor.

The induced hepatic stem cell to be used as the starting material in the present invention may be prepared from a cell of any mammalian origin. The mammal as the source of the cells may be exemplified by rat, mouse, guinea pig, rabbit, dog, cat, pig such as minipig, cow, horse, primates such as monkeys including a cynomologous monkey, and human, with rat, mouse, guinea pig, dog, cat, minipig, horse, cynomologous monkey, and human being preferred, and human is used with particular preference.

The mammalian cell to be used to prepare induced hepatic stem cells may be derived from any tissues. Examples include but are not limited to cells of organs such as the brain, liver, esophagus, stomach, duodenum, small intestine, large intestine, colon, pancreas, kidney, and lung, as well as cells of bone marrow fluid, muscle, fat tissue, peripheral blood, skin, and skeletal muscle.

It is also possible to use cells derived from tissues and body fluids that accompany childbirth such as cells derived from umbilical cord tissues (umbilical cord and umbilical cord blood), amnion, placenta and amniotic fluid; in particular, there may be used cells derived from tissues just after birth such as various tissues of neonates (e.g., neonatal skin).

The cells of the above-mentioned mammals that may be used include adult-derived cells, neonate-derived cells, neonatal skin-derived cells, cancerous individual's cells, etc.

In the previously filed international application (PCT/JP2001/000621; published as WO 2011/096223 on Aug. 11, 2011), the present inventor developed a method of preparing an induced hepatic stem cell that expresses not only genes characteristic of pluripotent stem cells such as embryonic stem cells but also genes characteristic of hepatocytes; the method was shown to be capable of providing an induced hepatic stem cell that expresses genes characteristic of hepatocytes in addition to their expressing genes characteristic of pluripotent stem cells such as embryonic stem cells. One characteristic of the induced hepatic stem cell is that it expresses at least the NANOG gene, the POU5F1 (OCT3/4) gene, and the SOX2 gene as selected from the group of the marker genes for embryonic stem cells and other pluripotent stem cells that are listed in the following Table 1.

TABLE 1

| GeneSymbol | GenbankAccession |
|---|---|
| ACVR2B | NM_001106 |
| CD24 | L33930 |
| CDH1 | NM_004360 |
| CYP26A1 | NM_057157 |
| DNMT3B | NM_175850 |
| DPPA4 | NM_018189 |
| EDNRB | NM_003991 |
| FLT1 | NM_002019 |
| GABRB3 | NM_000814 |
| GATA6 | NM_005257 |
| GDF3 | NM_020634 |
| GRB7 | NM_005310 |
| LIN28 | NM_024674 |
| NANOG | NM_024865 |
| NODAL | NM_018055 |
| PODXL | NM_005397 |
| POU5F1 | NM_002701 |
| SALL4 | NM_020436 |
| SOX2 | NM_003106 |
| TDGF1 | NM_003212 |
| TERT | NM_198253 |
| ZFP42 | NM_174900 |
| ZIC3 | NM_003413 |

In addition to expressing the above-mentioned genes which display the properties of embryonic stem cells, the induced hepatic stem cell to be used in the present invention is also characterized by having the properties of a hepatocyte or expressing genes associated with the properties of a hepatocyte. The properties of a hepatocyte that are to be possessed by the induced hepatic stem cell of the present invention are not particularly limited as long as they are characteristic of hepatocytes. The genes associated with the properties of a hepatocyte may be any gene that is characteristically expressed in a hepatocyte and which is associated with the properties of a hepatocyte such as a fetal hepatocyte or a mature hepatocyte (adult hepatocyte) (see the following Table 2). The induced hepatic stem cell to be used in the present invention may typically express genes characteristic of hepatocytes. Specific examples include the DLK1 gene, the AFP gene, the ALB gene, the AAT gene, the TTR gene, the FGG gene, the AHSG gene, the FABP1 gene, the RBP4 gene, the TF gene, the APOA4 gene, etc.

TABLE 2

| GeneSymbol | GenbankAccession |
|---|---|
| A2M | NM_000014 |
| ACE2 | NM_021804 |
| ACVRL1 | NM_000020 |
| ADAMTS9 | NM_182920 |
| AFAP1L2 | NM_001001936 |
| AFP | NM_001134 |
| AGT | NM_000029 |
| AHSG | NM_001622 |
| AK027294 | AK027294 |
| AK074614 | AK074614 |
| AK124281 | AK124281 |
| AK126405 | AK126405 |
| ALB | NM_000477 |
| ALDH1A1 | NM_000689 |
| ANXA8 | NM_001630 |
| APCDD1 | NM_153000 |
| APOA1 | NM_000039 |
| APOA2 | NM_001643 |
| APOA4 | NM_000482 |
| APOB | NM_000384 |
| AREG | NM_001657 |
| ART4 | NM_021071 |
| ASGR2 | NM_080912 |
| ATAD4 | NM_024320 |
| BC018589 | BC018589 |
| BMP2 | NM_001200 |
| BX097190 | BX097190 |
| C11orf9 | NM_013279 |
| C13orf15 | NM_014059 |
| C15orf27 | NM_152335 |
| C3 | NM_000064 |
| C5 | NM_001735 |
| CA414006 | CA414006 |
| CD163 | NM_004244 |
| CD1D | NM_001766 |
| CDX2 | NM_001265 |
| CILP | NM_003613 |
| CMKLR1 | NM_004072 |
| COL4A6 | NM_033641 |
| COLEC11 | NM_199235 |
| CXCL14 | NM_004887 |
| CXCR4 | NM_001008540 |
| CXCR7 | NM_020311 |
| DACH1 | NM_080759 |
| DENND2A | NM_015689 |
| DIO3 | NM_001362 |
| DLK1 | NM_003836 |
| DUSP6 | NM_001946 |
| ERP27 | NM_152321 |
| EVA1 | NM_144765 |
| F10 | NM_000504 |
| F2 | NM_000506 |
| FABP1 | NM_001443 |
| FGA | NM_021871 |
| FGA | NM_000508 |
| FGB | NM_005141 |
| FGG | NM_000509 |
| FLRT3 | NM_198391 |
| FMOD | NM_002023 |
| FOXA1 | NM_004496 |
| FTCD | NM_206965 |
| GATA4 | NM_002052 |
| GATM | NM_001482 |
| GDF10 | NM_004962 |
| GJB1 | NM_000166 |
| GLT1D1 | NM_144669 |
| GPRC5C | NM_022036 |
| GSTA3 | NM_000847 |
| GUCY1A3 | NM_000856 |
| H19 | NR_002196 |
| HHEX | NM_002729 |
| HKDC1 | NM_025130 |
| HMGCS2 | NM_005518 |
| HP | NM_005143 |
| HPR | NM_020995 |
| HPX | NM_000613 |
| HSD17B2 | NM_002153 |
| HTRA3 | NM_053044 |
| IGF2 | NM_001007139 |
| IL32 | NM_001012631 |
| INHBB | NM_002193 |
| ISX | NM_001008494 |
| KCNJ16 | NM_170741 |
| KYNU | NM_003937 |
| LAMC2 | NM_005562 |
| LGALS2 | NM_006498 |
| LHX2 | NM_004789 |
| LOC132205 | AK091178 |
| LOC285733 | AK091900 |
| M27126 | M27126 |
| MAF | AF055376 |
| MFAP4 | NM_002404 |
| MMP10 | NM_002425 |
| MTTP | NM_000253 |
| NGEF | NM_019850 |
| NGFR | NM_002507 |
| NRCAM | NM_005010 |
| NTF3 | NM_002527 |
| OLFML2A | NM_182487 |
| PAG1 | NM_018440 |
| PCSK6 | NM_002570 |
| PDK4 | NM_002612 |
| PDZK1 | NM_002614 |
| PLA2G12B | NM_032562 |
| PLG | NM_000301 |
| PRG4 | NM_005807 |
| PSMAL | NM_153696 |
| PTGDS | NM_000954 |
| PTHR1 | NM_000316 |
| RASD1 | NM_016084 |
| RBP4 | NM_006744 |
| RNF43 | NM_017763 |
| RRAD | NM_004165 |
| S100A14 | NM_020672 |
| SEPP1 | NM_005410 |
| SERINC2 | NM_178865 |
| SERPINA1 | NM_001002236 |
| SERPINA3 | NM_001085 |
| SERPINA5 | NM_000624 |
| SH3TC1 | NM_018986 |
| SLC13A5 | NM_177550 |
| SLC40A1 | NM_014585 |
| SLC5A9 | NM_001011547 |
| SLCO2B1 | NM_007256 |
| SLPI | NM_003064 |
| SPARCL1 | NM_004684 |
| SPON1 | NM_006108 |
| ST8SIA1 | NM_003034 |
| STARD10 | NM_006645 |
| STMN2 | S82024 |
| TDO2 | NM_005651 |
| TF | NM_001063 |
| TMC6 | NM_007267 |
| TMEM16D | NM_178826 |
| TSPAN15 | NM_012339 |
| TTR | NM_000371 |
| UBD | NM_006398 |
| UGT2B11 | NM_001073 |
| UGT2B7 | NM_001074 |
| UNC93A | NM_018974 |
| VCAM1 | NM_001078 |
| VIL1 | NM_007127 |
| VTN | NM_000638 |
| WFDC1 | NM_021197 |

The induced hepatic stem cell is preferably subjected to a step of bringing the cells mentioned above to such a state that gene products of the POU5F1 (OCT3/4) gene, the KLF4 gene, and the SOX2 gene which are necessary for inducing the differentiation into the induced hepatic stem cell will be present to ensure that the intracellular relative abundance of the gene product of the POU5F1 (OCTt3/4) gene is greater than that of the gene product of the SOX2 gene. One example of this step is a gene transfer that is performed to provide a higher ratio of the POU5F1 (OCT3/4) gene than the OX2 gene. The gene symbols for the POU5F1 (OCT3/4) gene, the KLF4 gene, and the SOX2 gene, as well as the corresponding Genbank accession numbers are given in Table 3.

TABLE 3

| GeneSymbol | GenbankAccession |
|---|---|
| KLF4 | NM_004235 |
| POU5F1 | NM_002701 |
| SOX2 | NM_003106 |

To prepare the induced hepatic stem cell, one or more of the genes known in induction techniques for giving rise to induced pluripotent stem cells, or gene products thereof (e.g. proteins and mRNAs) or agents, etc. can be expressed in, introduced into or added to the aforementioned mammalian cell. If necessary, the amounts of vectors to be introduced into the aforementioned mammalian cell, the amounts of genes to be introduced, the amounts of gene products to be added to media, and other parameters may be so adjusted as to ensure that the gene product of the POU5F1 gene has a greater intracellular relative abundance than the gene product of the SOX2 gene.

In the process of preparing the induced hepatic stem cell to be used in the present invention, the efficiency of induction of differentiation into the induced hepatic stem cell may be increased by adding known agents, compounds and antibodies as inducers of induced pluripotent stem cells to the media used to induce the differentiation into the induced hepatic stem cell of the present invention. These agents, compounds and antibodies are exemplified by inhibitors including: three low-molecular weight inhibitors of FGF receptor tyrosine kinase, MEK (mitogen activated protein kinase)/ERK (extracellular signal regulated kinases 1 and 2) pathway, and GSK (Glycogen Synthase Kinase) 3 [SU5402, PD184352, and CHIR99021], two low-molecular weight inhibitors of MEK/ERK pathway and GSK3 [PD0325901 and CHIR99021], a low-molecular weight compound as an inhibitor of the histone methylating enzyme G9a [BIX-01294 (BIX)], azacitidine, trichostatin A (TSA), 7-hydroxyflavone, lysergic acid ethylamide, kenpaullone, an inhibitor of TGF-β receptor I kinase/activin-like kinase 5 (ALK5) [EMD 616452], inhibitors of TGF-β receptor 1 (TGFBR1) kinase [E-616452 and E-616451], an inhibitor of Src-family kinase [EI-275], thiazovivin, PD0325901, CHIR99021, SU5402, PD184352, SB431542, anti-TGF-β neutralizing antibody, TGF-β inhibitor A-83-01, Nr5a2, a p53 inhibiting compound, siRNA against p53, an inhibitor of p53 pathway, and the like.

In the process of preparing the induced hepatic stem cell to be used in the present invention, it is also possible to use a microRNA which is used to prepare induced pluripotent stem cells, in order to increase the efficiency of induction of differentiation into the induced hepatic stem cell.

The step of inducing the differentiation of the aforementioned mammalian cell into an induced hepatic stem cell or an induced hepatic progenitor cell may involve the use of various inhibitors or antibodies that will inhibit or neutralize the activity of TGF-β or the like and which are to be added to the medium for culturing the induced hepatic stem cell of the present invention. Exemplary TGF-β inhibitors include TGF-β signaling inhibitors such as an ALK inhibitor (e.g. A-83-01), a TGF-β RI inhibitor, and a TGF-β RI kinase inhibitor.

These components are preferably added to the medium to be used in the step of inducing the differentiation of the aforementioned mammalian cell into an induced hepatic stem cell.

The induced hepatic stem cell having the features described above is characterized in that it can be subjected to expansion culture or passage culture for at least 3 days, preferably at least 14 days, and more preferably at least a month.

In the previous case of culturing stem cells such as embryonic stem cells, induced pluripotent stem cells and induced hepatic stem cells, various inhibitors or antibodies that can inhibit or neutralize the activity of TGF-β or the like have been added to media in order to ensure that no differentiation will occur even if they are cultured for longer than a month. However, it has been found that, in the present invention, highly efficient hepatic differentiation can be accomplished by culturing stem cells such as embryonic stem cells, induced pluripotent stem cells and induced hepatic stem cells in a culture medium supplemented with various inhibitors or antibodies that can inhibit or neutralize the activity of TGF-β or the like (which are collectively referred to as TGF-β inhibitors in the present invention). It has also been found that, in a preferred embodiment, induced hepatic stem cells undergo highly efficient differentiation into induced hepatic progenitor cells if they are cultured in a culture medium supplemented with the aforementioned TGF-β inhibitor. Specifically, culture in the presence of an added TGF-β inhibitor can be realized by adding a TGF-β inhibitor to a culture medium for use in culturing embryonic stem cells or induced pluripotent stem cells. The TGF-β inhibitor to be used in the present invention refers to any agent for inhibiting TGF-β functions or signal transduction by TGF-β and it may be in various forms including low-molecular weight compounds, antibodies, or antisense compounds.

Typical examples of the TGF-β inhibitor that can be used in the present invention include the following.

### <Low-Molecular Weight Compounds>

### TGF-β RI Kinase Inhibitor IX (ALK4, 5 and 7 Inhibitor), A-83-01

### (3-(6-methyl-2-pyridinyl)-N-phenyl-4-(4-quinolinyl)-1H-pyrazolo-1-carbothioamide)

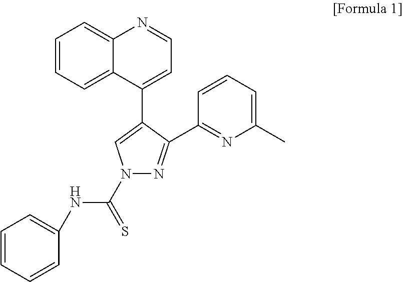 [SMILES: Cc1cccc(-c2nn(C(=S)Nc3ccccc3)cc2-c2ccnc3ccccc23)n1]

### TGF-β RI Kinase Inhibitor 1616451

### (3-(pyridin-2-yl)-4-(4-quinonyl)-1H-pyrazole)

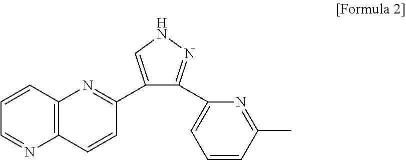 [SMILES: Cc1cccc(-c2n[nH]cc2-c2ccc3ncccc3n2)n1]

### LDN193189

### (4-(6-(4-(piperazin-1-yl)phenyl)pyrazolo[1,5-a]pyrimidin-3-yl)quinoline)

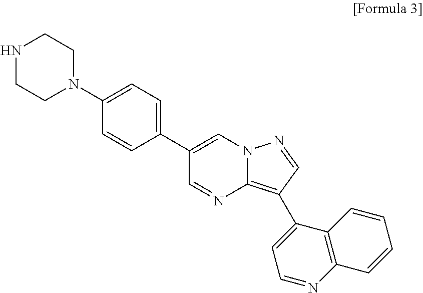 [SMILES: c1ccc2c(-c3cnn4cc(-c5ccc(N6CCNCC6)cc5)cnc34)ccnc2c1]

### TGF-β RI Kinase Inhibitor VI, SB431542

### (4-[4-(1,3-benzodioxol-5-yl)-5-pyridin-2-yl-1H-imidazol-2-yl]benzamide)

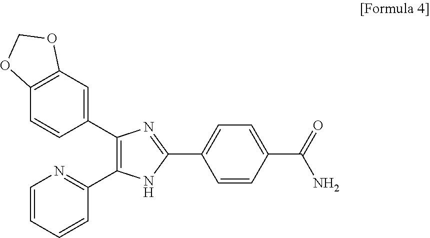 [SMILES: NC(=O)c1ccc(-c2nc(-c3ccc4c(c3)OCO4)c(-c3ccccn3)[nH]2)cc1]

### TGF-β Type I Receptors (ALK4, ALK5 and ALK7) Inhibitor, SB-505124

### (2-(5-benzo[1,3]dioxol-5-yl-2-tert-butyl-3H-imidazol-4-yl)-6-methylpyridine hydrochloride hydrate)

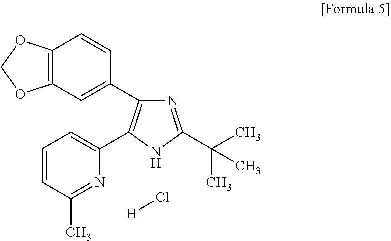

### TGF-β RI Kinase Inhibitor V 616456, SD-208

### ([(2-(5-chloro-2-fluorophenyl)pteridin-4-yl]pyridin-4-yl-amine)

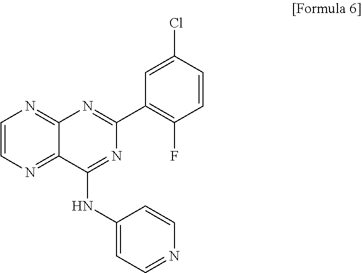 [SMILES: Fc1ccc(Cl)cc1-c1nc(Nc2ccncc2)c2nccnc2n1]

### SB-525334

### (6-[2-(1,1-dimethylethyl)-5-(6-methyl-2-pyridinyl)-1H-imidazol-4-yl]quinoxaline)

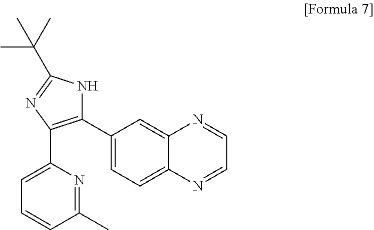 [SMILES: Cc1cccc(-c2nc(C(C)(C)C)[nH]c2-c2ccc3nccnc3c2)n1]

### LY-364947

### (4-[3-(2-pyridinyl)-1H-pyrazol-4-yl]-quinoline)

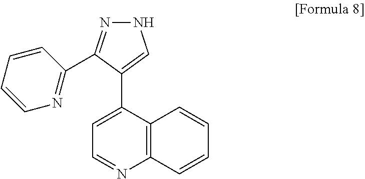 [SMILES: c1ccc(-c2n[nH]cc2-c2ccnc3ccccc23)nc1]

TGF-β RI Kinase Inhibitor, LY2157299

### (4-[2-(6-methyl-pyridin-2-yl)-5,6-dihydro-4H-pyrrolo[1,2-b]pyrazol-3-yl]-quinoline-6-carboxylic acid amide)

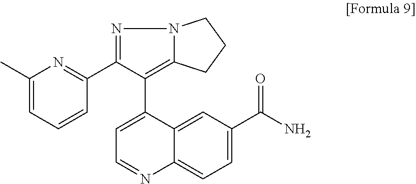 [SMILES: Cc1cccc(-c2nn3c(c2-c2ccnc4ccc(C(N)=O)cc24)CCC3)n1]

### TGF-β RI Kinase Inhibitor II 616452

### (2-(3-(6-methylpyridin-2-yl)-1H-pyrazol-4-yl)-1,5-naphthyridine)

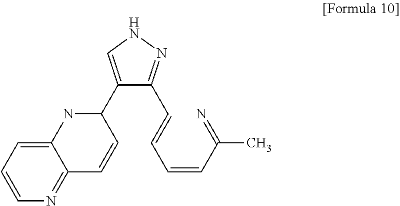 [SMILES: CC(=N)/C=C\C=C\c1n[nH]cc1C1C=Cc2ncccc2N1]

### TGF-β RI Kinase Inhibitor III 616453

### (2-(5-benzo[1,3]dioxol-4-yl-2-tert-butyl-1H-imidazol-4-yl)-6-methylpyridine, HCl)

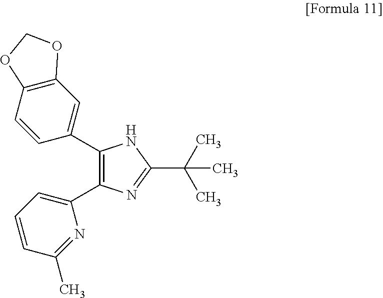 [SMILES: Cc1cccc(-c2nc(C(C)(C)C)[nH]c2-c2ccc3c(c2)OCO3)n1]

### TGF-β RI Kinase Inhibitor IX 616463

### (4-((4-((2,6-dimethylpyridin-3-yl)oxy)pyridin-2-yl)amino)benzenesulfonamide)

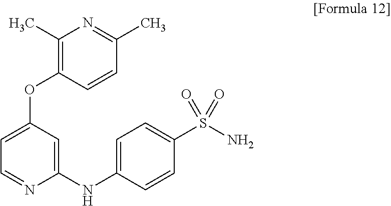 [SMILES: Cc1ccc(Oc2ccnc(Nc3ccc(S(N)(=O)=O)cc3)c2)c(C)n1]

### TGF-β RI Kinase Inhibitor VII 616458

### (1-(2-((6,7-dimethoxy-4-quinolyl)oxy)-4,5-dimethylphenyl)-1-ethanone)

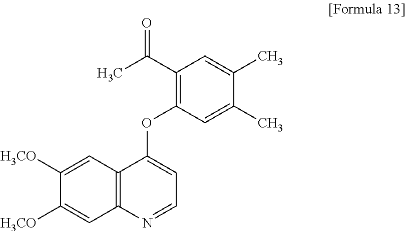 [SMILES: COc1cc2nccc(Oc3cc(C)c(C)cc3C(C)=O)c2cc1OC]

### TGF-β RI Kinase Inhibitor VIII 616459

### (6-(2-tert-butyl-5-(6-methyl-pyridin-2-yl)-1H-imidazol-4-yl)-quinoxaline)

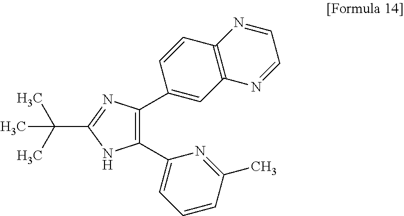 [SMILES: Cc1cccc(-c2[nH]c(C(C)(C)C)nc2-c2ccc3nccnc3c2)n1]

### <Antisense Oligonucleotides>

AP12009 (TGF-β2 antisense compound “Trabedersen”)
Belagenpumatucel-L (TGF-β2 antisense gene modified allogenic tumor cell vaccine)

### <Antibodies>

CAT-152 (Glaucoma-lerdelimumab (anti-TGF-β-2 monoclonal antibody))
CAT-192 (Metelimumab (human IgG4 monoclonal antibody which neutralizes TGFβ1))
GC-1008 (anti-TGF-β monoclonal antibody).

Among these TGF-β inhibitors, A-83-01 (3-(6-methyl-2-pyridinyl)-N-phenyl-4-(4-quinolinyl)-1H-pyrazolo-1-carbothioamide) as TGF-β RI Kinase Inhibitor IX (ALK4, 5 and 7 Inhibitor) is preferably used in the present invention. This is a selective inhibitor of type I TGF-β/activin receptor-like kinase (ALK5), type I activin/Nodal receptor-like kinase (ALK4) or type I Nodal receptor-like kinase (ALK7) and inhibits phosphorylation of Smad2/3 or TGF-β induced epithelial-mesenchymal transformation; A-83-01 is known to exert little or no effect on type I receptor for the osteogenic factor, p38 MAP kinase, or the extracellular signal regulated kinase; it has also been reported that A-83-01, if added to a rat iPS cell culture medium, allows uniform proliferation and prolonged culture of rat iPS cells without differentiation; A-83-01 also blocks phosphorylation of Smad2 and inhibits TGF-β induced epithelial-to-mesenchymal transition. As a TGF-β inhibitor, A-83-01 selectively inhibits ALK 4, ALK5 or ALK7 (with respective IC₅₀ values of 12, 45 and 7.5 nM). It has been known in the art concerned that by using this TGF-β inhibitor, rat iPS cells can be cultured uniformly over a prolonged period without differentiation.

Culture in the presence of the TGF-β inhibitor according to the method of the present invention is preferably performed in the absence of bFGF. By culturing induced hepatic stem cells under such conditions that the culture medium does not contain bFGF, hepatic differentiation into induced hepatic progenitor cells or hepatocytes through culture in the presence of the TGF-β inhibitor is promoted.

In another preferred embodiment, culture in the presence of the TGF-β inhibitor according to the method of the present invention may be performed in the presence of a substance selected from those having a steroid skeleton, a fatty acid and serum. The compound having a steroid skeleton may be exemplified by steroid hormones, bile acid, cholesterol, and synthetic steroids such as dexamethasone. By culturing induced hepatic stem cells in the presence of a substance selected from among compounds having a steroid skeleton, fatty acids or serum, hepatic differentiation into induced hepatic progenitor cells or hepatocytes through culture in the presence of the TGF-β inhibitor is promoted.

In yet another preferred embodiment, culture in the presence of the TGF-β inhibitor according to the method of the present invention may be performed in the absence of a feeder cell. By culturing induced hepatic stem cells or induced hepatic progenitor cells in the absence of a feeder cell, differentiation of the stem cells into hepatocytes through culture in the presence of the TGF-β inhibitor is promoted.

In still another preferred embodiment, culture in the presence of the TGF-β inhibitor according to the method of the present invention may be performed on a coated culture dish. By culturing induced hepatic stem cells or induced hepatic progenitor cells on a coated culture dish, differentiation of the induced hepatic stem cells or induced hepatic progenitor cells into hepatocytes through culture in the presence of the TGF-β inhibitor is promoted. Exemplary coating material that can be used in the present invention include a matrigel coat, collagen coat, gelatin coat, laminin coat, fibronectin coat, etc. with a matrigel coat being preferred.

The present invention is characterized by performing the step of culturing a stem cell, as selected from among induced hepatic stem cells or induced hepatic progenitor cells, for 1-4 weeks in the presence of any one of the TGF-β inhibitors described above.

To perform this step, there can be employed culture media that permit the expansion culture or passage culture of embryonic stem cells, pluripotent stem cells, and the like. Examples of such culture media include, but are not limited to, an ES medium [40% Dulbecco's modified Eagle medium (EMEM), 40% F12 medium (Sigma), 2 mM L-glutamine or GlutaMAX (Sigma), 1% non-essential amino acid (Sigma), 0.1 mM β-mercaptoethanol (Sigma), 15-20% Knockout Serum Replacement (Invitrogen), 10 μg/ml of gentamicin (Invitrogen), and 4-10 ng/ml of bFGF (FGF2) factor] (hereinafter referred to as ES medium), a conditioned medium that is the supernatant of a 24-hr culture of mouse embryonic fibroblasts (hereinafter referred to as MEF) on an ES medium lacking 0.1 mM β-mercaptoethanol and which is supplemented with 0.1 mM β-mercaptoethanol and 10 ng/ml of bFGF (FGF2) (this medium is hereinafter referred to as MEF conditioned ES medium), an optimum medium for iPS cells (iPSellon), an optimum medium for feeder cells (iPSellon), StemPro (registered trademark) hESC SFM (Invitrogen), mTeSR1 (STEMCELL Technologies/VERITAS), an animal protein free, serum-free medium for the maintenance of human ES/iPS cells, named TeSR2 [ST-05860] (STEMCELL Technologies/VERITAS), a medium for primate ES/iPS cells (ReproCELL), ReproStem (ReproCELL), ReproFF (ReproCELL), and ReproFF2 (ReproCELL). For human cells, media suitable for culturing human embryonic stem cells and pluripotent stem cells are preferably used.

As the techniques for effecting expansion culture or passage culture of induced hepatic stem cells or induced hepatic progenitor cells in the present invention, any of the methods commonly used by the skilled artisan to culture embryonic stem cells, pluripotent stem cells, and the like may be used. For example, after removing culture medium from the cultured cells and washing the cells with PBS(−), a dissociation solution is added and after standing for a given period, a D-MEM (high glucose) medium supplemented with 1× antibiotic-antimycotic and 10% FBS is added, the mixture is centrifuged, and the supernatant is removed; thereafter, 1× antibiotic-antimycotic, mTeSR, and 10 μM Y-27632 are added and the cell suspension is plated on an MEF-seeded, matrigel-, gelatin- or collagen-coated culture dish for effecting passage culture.

In the method of the present invention for differentiating an induced hepatic stem cell into induced hepatic progenitor cells or hepatocytes, the induced hepatic stem cell, before it is cultured in the presence of a TGF-β inhibitor, may be subjected to preliminary culture in a pluripotent stem cell culture medium in the presence of a feeder cell and only then the induced hepatic stem cell is cultured in the presence of a TGF-β inhibitor. As a result of this preliminary culture, the induced hepatic stem cell is brought into a preparatory stage for differentiation into induced hepatic progenitor cells or hepatocytes.

The culture described above induces differentiation of the induced hepatic stem cell into induced hepatic progenitor cells, and by further continuing the culture, differentiation of the induced hepatic progenitor cells into hepatocytes is induced.

As already mentioned, the induced hepatic stem cell that can be used in the present invention is characterized in that it expresses at least the POU5F1 (OCT3/4) gene, the NANOG gene, and the SOX2 gene as selected from the group of the genes listed in Table 1 and it is also characterized by induced expression of the genes listed in Table 2. By culturing this induced hepatic stem cell in the presence of a TGF-β inhibitor in accordance with the method of the present invention, differentiation into induced hepatic progenitor cells is first induced. The induced hepatic progenitor cell is characterized in that the expression of the hepatic stem/progenitor cell marker DLK1 or AFP gene as a gene associated with the properties of hepatocytes is increased markedly and that the expression of the hepatocyte markers ALB gene, AAT gene, TTR gene, FGG gene, AHSG gene, FABP1gene, RBP4 gene, TF gene, APOA4 gene, etc. is also increased markedly. The induced hepatic progenitor cell is also characterized in that the genes listed in Table 1 (at least the POU5F1 (OCT3/4) gene, the NANOG gene, the SOX2 gene, etc.) that have been expressed in the induced hepatic stem cell are expressed in the induced hepatic stem cell in smaller amounts ranging from about a tenth to a hundredth of the initial value.

In the present invention, the induced hepatic progenitor cells obtained by the above-described method are further cultured continuously to induce differentiation into hepatocytes. The thus obtained hepatocytes are characterized in that the genes listed in Table 1 which were expressed in the induced hepatic stem cell in amounts substantially comparable (⅛-8 times) to the levels expressed in the induced pluripotent stem cells are expressed in the hepatocyte in amounts even much smaller than the levels expressed in the induced hepatic stem cell, or their expression is substantially absent, and the hepatocytes are also characterized in that among the genes listed in Table 2 the expression of which was markedly induced in the induced hepatic progenitor cells, the hepatic stem/progenitor cell marker DLK1 or AFP gene is markedly decreased or substantially absent whereas the expression of the hepatocyte markers ALB gene, AAT gene, TTR gene, FGG gene, AHSG gene, FABP1gene, RBP4 gene, TF gene, APOA4 gene, etc. is increased even more markedly. It is also within the scope of the present invention that as the differentiation of the induced hepatic stem cell into induced hepatic progenitor cells is induced, at least one gene selected from among the SOX17 gene, the FOXA2 gene and the GATA4 gene which are characteristic of endodermal cells may become expressed, and as the differentiation of the induced hepatic progenitor cells into hepatocytes is induced, the expression of the genes listed in the following Table 4 is induced.

TABLE 4

| GeneSymbol | GenbankAccession |
|---|---|
| ABCB1 | NM_000927 |
| ABCB11 | NM_003742 |
| ABCB4 | NM_018850 |
| ABCC1 | NM_019862 |
| ABCC2 | NM_000392 |
| ABCC3 | NM_003786 |
| ACTB | NM_001101 |
| AHR | NM_001621 |
| ARNT | NM_001668 |
| BAAT | NM_001701 |
| COMT | NM_000754 |
| CYP1A1 | NM_000499 |
| CYP1A2 | NM_000761 |
| CYP1B1 | NM_000104 |
| CYP2A13 | NM_000766 |
| CYP2A6 | NM_000762 |
| CYP2A7 | NM_000764 |
| CYP2B6 | NM_000767 |
| CYP2C18 | NM_000772 |
| CYP2C19 | NM_000769 |
| CYP2C8 | NM_000770 |
| CYP2C9 | NM_000771 |
| CYP2D6 | NM_000106 |
| CYP2E1 | NM_000773 |
| CYP2F1 | NM_000774 |
| CYP2J2 | NM_000775 |
| CYP3A4 | NM_017460 |
| CYP3A5 | NM_000777 |
| CYP3A5 | AF355801 |
| CYP3A7 | NM_000765 |
| CYP4A11 | NM_000778 |
| CYP4B1 | NM_000779 |
| CYP4F11 | NM_021187 |
| CYP4F12 | NM_023944 |
| CYP4F2 | NM_001082 |
| CYP4F3 | AB002454 |
| CYP4F8 | NM_007253 |
| EEF1A1 | NM_001402 |
| ENDOG | NM_004435 |
| GAPDH | NM_002046 |
| GSTA1 | NM_145740 |
| GSTA2 | NM_000846 |
| GSTA3 | NM_000847 |
| GSTA4 | NM_001512 |
| GSTA5 | NM_153699 |
| GSTM1 | NM_146421 |
| GSTM2 | NM_000848 |
| GSTM3 | NM_000849 |
| GSTM4 | NM_147148 |
| GSTM5 | NM_000851 |
| GSTP1 | NM_000852 |
| GSTT1 | NM_000853 |
| GSTT2 | NM_000854 |
| GSTZ1 | NM_145870 |
| NAT1 | NM_000662 |
| NAT2 | NM_000015 |
| NR1H4 | NM_005123 |
| NR1I2 | NM_003889 |
| NR1I3 | NM_005122 |
| PPARA | NM_005036 |
| PPARA | L02932 |
| PPARD | NM_006238 |
| PPARG | NM_138711 |
| RPL13 | NM_033251 |
| RPS18 | NM_022551 |
| RXRA | NM_002957 |
| RXRB | NM_021976 |
| RXRG | NM_006917 |
| SLC10A1 | NM_003049 |
| SLC10A2 | NM_000452 |
| SLC16A1 | NM_003051 |
| SLC17A1 | NM_005074 |
| SLC22A1 | NM_153187 |
| SLC22A10 | NM_001039752 |
| SLC22A11 | AK075127 |
| SLC22A11 | NM_018484 |
| SLC22A2 | NM_003058 |
| SLC22A3 | NM_021977 |
| SLC22A4 | NM_003059 |
| SLC22A5 | NM_003060 |
| SLC22A6 | NM_153277 |
| SLC22A7 | NM_153320 |
| SLC22A8 | NM_004254 |
| SLC22A9 | NM_080866 |
| SLCO1A2 | NM_005075 |
| SLCO1A2 | NM_134431 |
| SLCO1B1 | NM_006446 |
| SLCO1B3 | NM_019844 |
| SLCO1C1 | NM_017435 |
| SLCO2A1 | NM_005630 |
| SLCO2B1 | NM_007256 |
| SLCO3A1 | XM_001132480 |
| SLCO3A1 | NM_013272 |
| SLCO4A1 | NM_016354 |
| SLCO4C1 | NM_180991 |
| SULT1A1 | NM_177529 |
| SULT1A2 | NM_177528 |
| SULT1A3 | AK094769 |
| SULT1A4 | NM_001017389 |
| SULT1B1 | D89479 |
| SULT1B1 | NM_014465 |
| SULT1C2 | NM_176825 |
| SULT1C4 | NM_006588 |
| SULT1E1 | NM_005420 |
| SULT2A1 | NM_003167 |
| SULT2B1 | NM_004605 |
| SULT4A1 | NM_014351 |
| TPMT | NM_000367 |
| UGT1A6 | NM_001072 |
| UGT1A8 | NM_019076 |
| UGT2A1 | NM_006798 |
| UGT2B10 | NM_001075 |
| UGT2B11 | NM_001073 |
| UGT2B15 | NM_001076 |
| UGT2B17 | NM_001077 |
| UGT2B28 | NM_053039 |
| UGT2B4 | NM_021139 |
| UGT2B7 | NM_001074 |

The experimental results associated with the present invention may schematically be summarized in the following Table 5.

TABLE 5

| | Induced hepatic | Induced hepatic | |
|---|---|---|---|
| | stem cell | progenitor cell | Hepatocyte |
| Genes in | +++ | + | − |
| Table 1 | | | |
| Genes in | + | +++ | ++++ |
| Table 2 | | | |
| Genes in | ± | ± | +++ |
| Table 3 | | | |

To amplify the nucleic acid sequences of the genes of interest, the primers listed in the following Table 6 were used.

TABLE 6

| | | | | | Product | NCBI |
|---|---|---|---|---|---|---|
| | Primer | | | Primer | size | Accession |
| Group | name | 5′-sequence-3′ | Tm | Size | (bp) | No. |
| HKG | GAPDH-F | ggcctccaaggagtaagacc | 60.07 | 20 | 147 | NM_002046 |
| HKG | GAPDH-R | aggggtctacatggcaactg | 59.99 | 20 | | |
| ES cells | OCT3/4-F | agtgagaggcaacctggaga | 59.99 | 20 | 110 | NM_002701 |
| ES cells | OCT3/4-R | acactcggaccacatccttc | 59.97 | 20 | | |
| ES cells | SOX2-F | tggtacggtaggagctttgc | 60.27 | 20 | 80 | NM_003106 |
| ES cells | SOX2-R | tttttcgtcgcttggagact | 59.99 | 20 | | |
| ES cells | NANOG-F | cagtctggacactggctgaa | 60.02 | 20 | 149 | NM_024865 |
| ES cells | NANOG-R | ctcgctgattaggctccaac | 59.98 | 20 | | |
| Endoderm | SOX17-F | ctgccacttgaacagtttgg | 59.33 | 20 | 184 | NM_022454 |
| Endoderm | SOX17-R | cacacccaggacaacatttc | 58.83 | 20 | | |
| Endoderm | FOXA2-F | gagggctactcctccgtga | 60.36 | 19 | 144 | NM_021784 |
| Endoderm | FOXA2-R | gcccacgtacgacgacat | 60.57 | 18 | | |
| Endoderm | GATA4-F | gctccttcaggcagtgagag | 60.28 | 20 | 130 | NM_002052 |
| Endoderm | GATA4-R | gcccgtagtgagatgacagg | 60.68 | 20 | | |
| Hepatic SC | DLK1-F | atgctgcggaagaagaagaa | 60.10 | 20 | 94 | NM_003836 |
| Hepatic SC | DLK1-R | tggtcatgtcgatcttctcg | 59.79 | 20 | | |
| Hepatic TF | HNF1A-F | gagcaaagaggcactgatcc | 59.96 | 20 | 208 | NM_000545 |
| Hepatic TF | HNF1A-R | ctccagctctttgaggatgg | 59.94 | 20 | | |
| Hepatic TF | HNF4A-F | ctgtcccgacagatcacctc | 60.68 | 20 | 137 | NM_000457 |
| Hepatic TF | HNF4A-R | gggatgtacttggcccactc | 61.68 | 20 | | |
| Cholangiocyte | KRT7-F | agcaatgccctgagcttct | 60.11 | 19 | 160 | NM_005556.3 |
| Cholangiocyte | KRT7-R | gggtgggaatcttcttgtga | 59.90 | 20 | | |
| Cholangiocyte | KRT19-F | agcaggtccgaggttactga | 59.87 | 20 | 199 | NM_002276 |
| Cholangiocyte | KRT19-R | gctcactatcagctcgcaca | 60.32 | 20 | 199 | |
| Hepatocyte | ALB-F | aatgccctgtgcagaagact | 68.00 | 20 | 101 | NM_000477 |
| Hepatocyte | ALB-R | ctgtgcagcatttggtgact | 68.00 | 20 | | |
| Hepatic SC | AFP-F | aaatgcgtttctcgttgctt | 64.00 | 20 | 136 | NM_001134 |
| Hepatic SC | AFP-R | gccacaggccaatagtttgt | 68.00 | 20 | | |
| Hepatocyte | AAT-F | tctttgtgcctgttgctgtc | 68.00 | 20 | 93 | NM_000295 |
| Hepatocyte | AAT-R | taccacaggggctattcagg | 70.00 | 20 | | |
| Hepatocyte | TTR-F | gcatgcagaggtggtattca | 68.00 | 20 | 93 | NM_000371 |
| Hepatocyte | TTR-R | gccgtggtggaataggagta | 70.00 | 20 | | |
| Hepatocyte | FABP1-F | ctgcagagccaggaaaactt | 59.62 | 20 | 208 | NM_001443 |
| Hepatocyte | FABP1-R | tctcccctgtcattgtctcc | 60.05 | 20 | | |
| Hepatocyte | FGG-F | ccaaacaggctggagacg | 60.39 | 20 | 151 | NM_000509 |
| Hepatocyte | FGG-R | caacatggggtcttttgctc | 60.50 | 20 | | |
| Hepatocyte | RBP4-F | ggcagtacaggctgatcgtc | 60.83 | 20 | 172 | NM_006744 |
| Hepatocyte | RBP4-R | ctgagggaagatggggagag | 61.12 | 20 | | |
| Hepatocyte | TF-F | ctcgggcaacttttgtttgt | 60.15 | 20 | 167 | M_001063 |
| Hepatocyte | TF-R | ggagtgatgaggtggagcat | 60.08 | 20 | | |
| Hepatocyte | AHSG-F | ggggaggatcagacacttca | 60.50 | 20 | 219 | NM_001622 |
| Hepatocyte | AHSG-R | ataaccaccacccactctgc | 59.85 | 20 | | |
| Hepatocyte | APOA4-F | ggaacagctcaggcagaaac | 60.00 | 20 | 153 | NM_000482 |
| Hepatocyte | APOA4-R | agctcagggagggagagagt | 59.56 | 20 | | |
| Hepatic | HGF-F | ggacgcagctacaagggaac | 62.1 | 20 | 151 | NM_001010931 |
| Hepatic | HGF-R | cccctcgaggatttcgac | 60.56 | 18 | | |
| CYP | CYP1A2-F | tgttcaagcacagcaagaagg | 61.52 | 21 | 70 | NM_000761 |
| CYP | CYP1A2-R | tgctccaaagatgtcattgac | 58.71 | 21 | | |
| CYP | CYP3A4-F | gaaacacagatccccctgaa | 59.90 | 20 | 161 | NM_017460 |
| CYP | CYP3A4-R | ctggtgttctcaggcacaga | 60.02 | 20 | | |
| CYP | CYP2C9-F | ggacagagacgacaagcaca | 60.03 | 20 | 156 | NM_000771 |
| CYP | CYP2C9-R | catctgtgtagggcatgtgg | 59.98 | 20 | | |

Based on these results, the present invention can provide an induced hepatic progenitor cell by differentiation of the above-described induced hepatic stem cell which is cultured for 1-4 weeks in the presence of a TGF-β inhibitor. The induced hepatic progenitor cell is characterized by satisfying at least the following two requirements (1) and (2):

(1) it expresses the OCT3/4, SOX2 and NANOG genes which are marker genes for an embryonic stem cell; and
(2) it expresses DLK1 and AFP which are hepatic stem/progenitor cell markers, as well as ALB, AAT and TTR which are hepatocyte markers.

In a preferred embodiment of the present invention, the induced hepatic progenitor cell of the present invention may be a cell that expresses the hepatocyte markers FGG, AHSG, FABP1, RBP4, TF and APOA4 in addition to the above-mentioned markers. Thus, a preferred cell of the present invention is characterized by satisfying the following two requirements (1) and (2):

(1) it expresses the OCT3/4, SOX2 and NANOG genes which are marker genes for an embryonic stem cell; and
(2) it expresses DLK1 and AFP which are hepatic stem/progenitor cell markers, as well as ALB, AAT, TTR, FGG, AHSG, FABP1, RBP4, TF and APOA4 which are hepatocyte markers.

As it turned out, the genes listed in Table 1 which are characteristic of the induced hepatic stem cell (e.g., the POU5F1 (OCT3/4) gene, the NANOG gene, and the SOX2 gene) were expressed in the induced hepatic progenitor cell in amounts that were very small (ten to hundred times less) compared to their levels expressed in the embryonic stem cell or induced hepatic stem cell.

On the other hand, the induced hepatic progenitor cell is characterized by a marked increase in the expression of the genes listed in Table 2 which was induced in the induced hepatic stem cell. The genes listed in Table 2 are characterized in that the amounts of expression of the hepatic stem/progenitor cell markers DLK1 and AFP or the amounts of expression of the hepatocyte markers ALB, AAT, TTR FGG, AHSG, FABP1, RBP4, TF and APOA4 may be markedly increased, say, 10-50,000 times more, compared to their levels expressed in the embryonic stem cell or induced hepatic stem cell.

In addition to the genes listed in Table 2, genes associated with the properties of a hepatocyte, such as hepatocyte-associated marker genes, for example, biliary duct epithelial cell markers KRT7 and KRT19, hepatocyte transcription factors HNF1A and HNF4A, or hepatocyte growth factor HGF may be expressed in increased amounts in the induced hepatic progenitor cell of the present invention.

### EXAMPLES

### Example 1

### Induction of Hepatic Differentiation, and Preparation of Induced Hepatic Progenitor Cells, without Feeder Cells

In a 10 cm-diameter culture dish coated with Matrigel (at an amount of 60 μL Matrigel/6 mL PBS/dish for an hour), the human induced hepatic stem cells AFB1-1 (No. 377; about 50% confluence/dish), which were cocultured with feeder cells (about 1.5×10⁶ mouse embryonic fibroblasts (MEF)/dish) using the human ES/iPS cell medium (mTeSR1; STEMCELL Technologies) (supplemented with 100 ng/mL bFGF) and washed with PBS (−), were dissociated from the culture dish with a 0.25% trypsin/1 mM EDTA solution (Invitrogen; 25200-056) and suspended in the human ES/iPS cell medium (ReproStem; ReproCELL), then a tenth of the suspension was subjected to centrifugal washing (at 1,000 rpm for 5 minutes). The human induced hepatic stem cells were suspended in mTeSR1/Y-27632 (10 μM) and then seeded (at a density of about 4×10⁴ cells/1 mL medium/well) in a E-well plate coated with Matrigel (at an amount of 10 μL Matrigel/1 mL PBS/well for an hour). After about three hours, the medium was replaced with 2 mL of mTeSR1 (supplemented with 100 ng/mL bFGF) to culture the cells without feeder cells. In the present invention, the resultant cell sample is called “No. 390” (refer to Table 7A).

Three and five days after the seeding, the medium was replaced with a fresh medium of the same composition, and the cells were subjected to differentiation culture; thereafter, until 12 days after the seeding, the medium was replaced everyday to continue differentiation culture. Thirteen days after the seeding, the culture supernatant was subjected to measurement (SRL Inc.) for α-fetoprotein (AFP) which is a marker protein for fetal hepatocytes (marker protein for hepatic stem/progenitor cells and hepatoblasts, which is not expressed in mature hepatocytes), and 163 ng/mL of AFP was observed in No. 390 (refer to Table 8A).

The cells were lysed in 1 mL/well of a QIAzol reagent to prepare the total RNA from the cell lysate using the miRNeasy Mini Kit manufactured by Qiagen. The total RNA was subjected to quantitative RT-PCR using the SuperScript III First-Strand Synthesis System (18080-051), the Platinum SYBR Green qPCR SuperMix-UDG (for any instrument) (11733-038), and the ABI7300 RealTime PCR System, all manufactured by Invitrogen. The quantified genes were the hepatic progenitor cell markers (DLK1, AFP) and the hepatocyte markers (ALB, TTR, AAT).

According to the results of the quantitative RT-PCR, the expression levels of these markers in the human induced hepatic progenitor cells (No. 390) increased by 126 to 675 times (i.e., 264 and 126 times for the hepatic stem/progenitor cell markers DLK1 and AFP, respectively, and 19, 14 and 675 times for the hepatocyte markers ALB, AAT and TTR, respectively, as compared with the respective marker expression levels in No. 377 being taken as 1). In other words, as compared with the human induced hepatic stem cells, the human induced hepatic progenitor cells increased in the expression levels of the hepatic stem/progenitor cell markers (DLK1, AFP) and the hepatocyte markers (ALB, AAT, TTR) (refer to Table 8A).

As is evident from the above-noted results, the culture procedure without feeder cells was suitable for effectively preparing human induced hepatic progenitor cells from human induced hepatic stem cells.

### Example 2

### Induction of Hepatic Differentiation, and Preparation of Induced Hepatic Progenitor Cells, in the Absence of bFGF

In a 10 cm-diameter culture dish coated with Matrigel (at an amount of 60 μL Matrigel/6 mL PBS/dish for an hour), the human induced hepatic stem cells AFB1-1 (No. 377; about 50% confluence/dish), which were cocultured with feeder cells (about 1.5×10⁶ mouse embryonic fibroblasts (MEF)/dish) using the human ES/iPS cell medium (mTeSR1; STEMCELL Technologies) (supplemented with 100 ng/mL bFGF) and washed with PBS (−), were dissociated from the culture dish with a 0.25% trypsin/1 mM EDTA solution and suspended in the human ES/iPS cell medium (ReproStem; ReproCELL), then a tenth of the suspension was subjected to centrifugal washing (at 1,000 rpm for 5 minutes). The human induced hepatic stem cells were suspended in mTeSR1/Y-27632 (10 μM) and then seeded (at a density of about 4×10⁴ cells/1 mL medium/well) in a 6-well plate coated with Matrigel (at an amount of 10 μL Matrigel/1 mL PBS/well for an hour). After about three hours, the medium was replaced with 2 mL of aFGF [10 ng/mL]/ReproStem (bFGF-free) to culture the cells without feeder cells. In the present invention, the resultant cell sample is called “No. 391” (refer to Table 7A).

Three and five days after the seeding, the medium was replaced with a fresh medium of the same composition, and the cells were subjected to differentiation culture; thereafter, until 12 days after the seeding, the medium was replaced everyday to continue differentiation culture. Thirteen days after the seeding, the culture supernatant was subjected to measurement (SRL Inc.) for α-fetoprotein (AFP) which is a marker protein for fetal hepatocytes (marker protein for hepatic stem/progenitor cells and hepatoblasts), and 3,300 ng/mL of AFP was observed in No. 391 (refer to Table 8A).

The cells were lysed in 1 mL/well of a QIAzol reagent to prepare the total RNA from the cell lysate using the miRNeasy Mini Kit manufactured by Qiagen. The total RNA was subjected to quantitative RT-PCR using the SuperScript III First-Strand Synthesis System (18080-051), the Platinum SYBR Green qPCR SuperMix-UDG (for any instrument) (11733-038), and the ABI7300 RealTime PCR System, all manufactured by Invitrogen. The quantified genes were the hepatic progenitor cell markers (DLK1, AFP) and the hepatocyte markers (ALB, TTR, AAT).

According to the results of the quantitative RT-PCR, the expression levels of these markers in the human induced hepatic progenitor cells (No. 391) increased by 220 to 3,910 times (i.e., 786 and 3,420 times for the hepatic stem/progenitor cell markers DLK1 and AFP, respectively, and 3,172,220 and 3,910 times for the hepatocyte markers ALB, AAT and TTR, respectively, as compared with the respective marker expression levels in No. 377 being taken as 1). In other words, as compared with the human induced hepatic stem cells, the human induced hepatic progenitor cells increased in the expression levels of the hepatic stem/progenitor cell markers (DLK1, AFP) and the hepatocyte markers (ALB, AAT, TTR) (refer to Table 8A).

As is evident from the above-noted results, the culture procedure in the presence of aFGF and substantially in the absence of bFGF was suitable for effectively preparing human induced hepatic progenitor cells from human induced hepatic stem cells.

### Example 3

### Induction of Hepatic Differentiation, and Preparation of Induced Hepatic Progenitor Cells, Using a TGF-β Signaling Inhibitor (1)

In a 10 cm-diameter culture dish coated with Matrigel (at an amount of 60 μL Matrigel/6 mL PBS/dish for an hour), the human induced hepatic stem cells AFB1-1 (No. 377; about 50% confluence/dish), which were cocultured with feeder cells (about 1.5×10⁶ mouse embryonic fibroblasts (MEF)/dish) using the human ES/iPS cell medium (mTeSR1; STEMCELL Technologies) (supplemented with 100 ng/mL bFGF) and washed with PBS (−), were dissociated from the culture dish with a 0.25% trypsin/1 mM EDTA solution and suspended in the human ES/iPS cell medium (ReproStem; ReproCELL), then a tenth of the suspension was subjected to centrifugal washing (at 1,000 rpm for 5 minutes). The human induced hepatic stem cells were suspended in mTeSR1/Y-27632 (10 μM) and then seeded (at a density of about 4×10⁴ cells/1 mL medium/well) in a 6-well plate coated with Matrigel (at an amount of 10 μL Matrigel/1 mL PBS/well for an hour). After about three hours, the medium was replaced with 2 mL of 0.1 μM A-83-01 (TOCRIS; Cat. No. 2939)/mTeSR1 (supplemented with 100 ng/mL bFGF) to culture the cells without feeder cells. In the present invention, the resultant cell sample is called “No. 393” (refer to Table 7A).

Three and five days after the seeding, the medium was replaced with a fresh medium of the same composition, and the cells were subjected to differentiation culture; thereafter, until 12 days after the seeding, the medium was replaced everyday to continue differentiation culture. Thirteen days after the seeding, the culture supernatant was subjected to measurement (SRL Inc.) for α-fetoprotein (AFP) which is a marker protein for fetal hepatocytes (marker protein for hepatic stem/progenitor cells and hepatoblasts, which is not expressed in mature hepatocytes), and 3,120 ng/mL of AFP was observed in No. 393 (refer to Table 8A).

The cells were lysed in 1 mL/well of a QIAzol reagent to prepare the total RNA from the cell lysate using the miRNeasy Mini Kit manufactured by Qiagen. The total RNA was subjected to quantitative RT-PCR using the SuperScript III First-Strand Synthesis System (18080-051), the Platinum SYBR Green qPCR SuperMix-UDG (for any instrument) (11733-038), and the ABI7300 RealTime PCR System, all manufactured by Invitrogen. The quantified genes were the hepatic progenitor cell markers (DLK1, AFP) and the hepatocyte markers (ALB, TTR, AAT).

According to the results of the quantitative RT-PCR, the expression levels of these markers in the human induced hepatic progenitor cells (No. 393) increased by 240 to 2,871 times (i.e., 404 and 1,791 times for the hepatic stem/progenitor cell markers DLK1 and AFP, respectively, and 1,925, 240 and 2,871 times for the hepatocyte markers ALB, AAT and TTR, respectively, as compared with the respective marker expression levels in No. 377 being taken as 1). In other words, as compared with the human induced hepatic stem cells, the human induced hepatic progenitor cells increased in the expression levels of the hepatic stem/progenitor cell markers (DLK1, AFP) and the hepatocyte markers (ALB, AAT, TTR) (refer to Table 8A).

As is evident from the above-noted results, the culture procedure in the presence of the TGF-β signaling inhibitor A-83-01 was suitable for effectively preparing human induced hepatic progenitor cells from human induced hepatic stem cells.

### Example 4

### Induction of Hepatic Differentiation, and Preparation of Induced Hepatic Progenitor Cells, in the Absence of Feeder Cells or bFGF and in the Presence of the TGF-β Inhibitor on a Matrigel-Coated Dish

In a 10 cm-diameter culture dish coated with Matrigel (at an amount of 60 μL Matrigel/6 mL PBS/dish for an hour), the human induced hepatic stem cells AFB1-1 (No. 377; about 50% confluence/dish), which were cocultured with feeder cells (about 1.5×10⁶ mouse embryonic fibroblasts (MEF)/dish) using the human ES/iPS cell medium (mTeSR1; STEMCELL Technologies) (supplemented with 100 ng/mL bFGF) and washed with PBS (−), were dissociated from the culture dish with a 0.25% trypsin/1 mM EDTA solution and suspended in the human ES/iPS cell medium (ReproStem; ReproCELL), then a tenth of the suspension was subjected to centrifugal washing (at 1,000 rpm for 5 minutes). The human induced hepatic stem cells were suspended in mTeSR1/Y-27632 (10 μM) and then seeded (at a density of about 4×10⁴ cells/1 mL medium/well) in a 6-well plate coated with Matrigel (at an amount of 10 μL Matrigel/1 mL PBS/well for an hour). After about three hours, the medium was replaced with 2 mL of 0.1 μM A-83-01/aFGF [10 ng/mL]/ReproStem (bFGF-free) to culture the cells without feeder cells. In the present invention, the resultant cell sample is called “No. 394” (refer to Table 7A).

Three and five days after the seeding, the medium was replaced with a fresh medium of the same composition, and the cells were subjected to differentiation culture; thereafter, until 12 days after the seeding, the medium was replaced everyday to continue differentiation culture. Thirteen days after the seeding, the culture supernatant was subjected to measurement (SRL Inc.) for α-fetoprotein (AFP) which is a marker protein for fetal hepatocytes (marker protein for hepatic stem/progenitor cells and hepatoblasts, which is not expressed in mature hepatocytes), and 14,400 ng/mL of AFP was observed in No. 394 (refer to Table 8A).

The cells were lysed in 1 mL/well of a QIAzol reagent to prepare the total RNA from the cell lysate using the miRNeasy Mini Kit manufactured by Qiagen. The total RNA was subjected to quantitative RT-PCR using the SuperScript III First-Strand Synthesis System (18080-051), the Platinum SYBR Green qPCR SuperMix-UDG (for any instrument) (11733-038), and the ABI7300 RealTime PCR System, all manufactured by Invitrogen. The quantified genes were the embryonic stem cell markers (OCT3/4 [POU5F1], SOX2, NANOG), the endoderm markers (SOX17, FOXA2, GATA4), the hepatic stem/progenitor cell markers (DLK1, AFP), the hepatocyte markers (ALB, TTR, AAT, FGG, AHSG, FABP1, RBP4, TF, APOA4), the hepatocyte transcription factors (HNF1A, HNF4A), the biliary duct epithelial cell marker (KRT7), and the hepatocyte growth factor (HGF).

The results of the quantitative RT-PCR are as follows. The human induced hepatic stem cells (No. 377) expressed the embryonic stem cell markers (OCT3/4 [POU5F1], SOX2, NANOG), the endoderm markers (SOX17, FOXA2, GATA4), the hepatic stem/progenitor cell markers (DLK1, AFP), the hepatocyte transcription factors (HNF1A, HNF4A), the hepatocyte markers (ALB, TTR, AAT, FGG, AHSG, FABP1, RBP4, TF, APOA4), the biliary duct epithelial cell marker (KRT7), and the hepatocyte growth factor (HGF). In the human induced hepatic progenitor cell sample (No. 394), the expression levels of the embryonic stem cell markers decreased to 1-8% (i.e, 0.07, 0.08, and 0.01 times for OCT3/4 (POU5F1), SOX2 and NANOG, respectively, as compared with the respective marker expression levels in No. 377 being taken as 1) and the expression levels of the endoderm markers decreased to 3-20% (i.e., 0.03, 0.19, and 0.02 times for SOX17, FOXA2 and GATA4, respectively, as compared with the respective marker expression levels in No. 377 being taken as 1), and the hepatocyte transcription factors (HNF1A, HNF4A) were also expressed (in amounts 0.88 and 0.39 times, as compared with the respective marker expression levels in No. 377 being taken as 1). However, the expression levels of the hepatic stem/progenitor cell markers and hepatocyte markers in No. 394 increased by 804 to 45,698 times (i.e., 804 and 12,812 times for DLK1 and AFP, respectively, and 45,698, 3,812, 9,113, 10,138, 14,079, 3,034, 4,326, 9,126 and 966 times for ALB, AAT, TTR, FGG, AHSG, FABP1, RBP4, TF and APOA4, respectively, as compared with the respective marker expression levels in No. 377 being taken as 1), and the expression levels of the biliary duct epithelial cell marker (KRT7) and HGF in No. 394 also increased (by 37.5 time for KRT7 and 11 times for HGF, as compared with the respective marker expression levels in No. 377 being taken as 1). That is to say, in the human induced hepatic progenitor cells, as compared with the human induced hepatic stem cells, the expression levels of the embryonic stem cell markers (OCT3/4 [POU5F1], SOX2, NANOG) decreased to not more than 10% and the endoderm markers (SOX17, FOXA2, GATA4) decreased to not more than 25%, respectively, and the expression levels of the hepatic stem/progenitor cell markers (DLK1, AFP) and the hepatocyte markers (ALB, AAT, TTR, FGG, AHSG, FABP1, RBP4, TF, APOA4) increased by 100 times or more, and the expression levels of the biliary duct epithelial cell marker (KRT7) and the hepatocyte growth factor (HGF) also increased by 10 times or more (refer to Table 8A).

As is evident from the above-noted results, the culture procedure in a Matrigel-coated dish without feeder cells using a medium that contains substantially no bFGF (at most 0.01 pg/mL even including that derived from Matrigel coat) but was supplemented with A-83-01 was suitable for effectively preparing human induced hepatic progenitor cells from human induced hepatic stem cells.

In this connection, the induced pluripotent stem cells expressed the embryonic stem cell markers (OCT3/4 [POU5F1], SOX2, NANOG) at the levels comparable to the human induced hepatic stem cells (i.e., levels that are ¼-4 times as compared to the levels of those cells), and did not substantially express the hepatic stem/progenitor cell markers (DLK1, AFP), the hepatocyte markers (ALB, AAT, TTR, FGG, AHSG, FABP1, RBP4, TF, APOA4), the biliary duct epithelial cell marker (KRT7), or the hepatocyte growth factor (HGF). Some induced pluripotent stem cells may express not only the embryonic stem cell markers but also any two or three of the above-noted genes due to expression disorder. However, no cell line has been reported that, like the human induced hepatic stem cells, expressed all of the hepatic stem/progenitor cell markers (DLK1, AFP), the hepatocyte markers (ALB, AAT, TTR, FGG, AHSG, FABP1, RBP4, TF, APOA4), the biliary duct epithelial cell marker (KRT7), and the hepatocyte growth factor (HGF).

### Example 5

### Induction of Hepatic Differentiation by Suspension (Three-Dimensional) Culture in the Absence of bFGF

In a 10 cm-diameter culture dish coated with Matrigel (at an amount of 15 μL Matrigel/6 mL PBS/dish), the human induced hepatic stem cells AFB 1-1 (No. 451; about 50% confluence/dish), which were cocultured with feeder cells (about 1.5×10⁶ mouse embryonic fibroblasts (MEF)/dish) using the human ES/iPS cell medium (mTeSR1; STEMCELL Technologies) (supplemented with 100 ng/mL bFGF) and washed with PBS (−), were dissociated from the culture dish with a 0.25% trypsin/1 mM EDTA solution and suspended in the human ES/iPS cell medium (ReproStem; ReproCELL), then the suspension was subjected to centrifugal washing (at 1,000 rpm for 5 minutes). The human induced hepatic stem cells were suspended in ReproStem (bFGF-free)/Y-27632 (5 μM) and then seeded (at a density of about 8×10⁴ cells/5 mL medium/well) in a low-attachment 6-well culture plate (Corning; Cat. No. 3471) to culture the cells without feeder cells. In the present invention, the resultant cell sample is called “No. 472” (refer to Table 7B).

Six days after the seeding, the cells were recovered centrifugally and then suspended in 2 mL of a fresh ReproStem (bFGF-free)/Y-27632 (5 μM) to continue hepatic differentiation culture in the above culture plate. On the following day, the centrifuged culture supernatant was subjected to measurement (SRL Inc.) for α-fetoprotein (AFP) which is a marker protein for fetal hepatocytes (marker protein for hepatic stem/progenitor cells and hepatoblasts, which is not expressed in mature hepatocytes), and 349 ng/mL of AFP was observed in No. 472. The cell pellets were lysed in 1 mL/well of a QIAzol reagent (refer to Table 8B).

As described above, hepatic differentiation was induced by suspension (three-dimensional) culture in the absence of bFGF to effectively prepare human induced hepatic progenitor cells or human hepatocytes from human induced hepatic stem cells.

### Example 6

### Induction of Hepatic Differentiation by Suspension (Three-Dimensional) Culture in the Presence of the TGF-β Inhibitor

In a 10 cm-diameter culture dish coated with Matrigel (at an amount of 15 μL Matrigel/6 mL PBS/dish), the human induced hepatic stem cells AFB 1-1 (No. 451; about 50% confluence/dish), which were cocultured with feeder cells (1.5×10⁶ mouse embryonic fibroblasts (MEF)/dish) using the human ES/iPS cell medium (mTeSR1; STEMCELL Technologies) (supplemented with 100 ng/mL bFGF) and washed with PBS (−), were dissociated from the culture dish with a 0.25% trypsin/1 mM EDTA solution and suspended in the human ES/iPS cell medium (ReproStem; ReproCELL), then the suspension was subjected to centrifugal washing (at 1,000 rpm for 5 minutes). The human induced hepatic stem cells were suspended in ReproStem (bFGF-free)/Y-27632 (5 μM) supplemented with 0.1 μM TGF-β inhibitor (A-83-01), and were then seeded (at a density of about 8×10⁴ cells/5 mL medium/well) in a low-attachment 6-well culture plate (Corning; Cat. No. 3471) to culture the cells without feeder cells. In the present invention, the resultant cell sample is called “No. 473” (refer to Table 7B).

Six days after the seeding, the cells were recovered centrifugally and then suspended in 2 mL of a fresh ReproStem (bFGF-free)/Y-27632 (5 μM) supplemented with 0.1 μM TGF-β inhibitor (A-83-01), to continue hepatic differentiation culture in the above culture plate. On the following day, the centrifuged culture supernatant was subjected to measurement (SRL Inc.) for α-fetoprotein (AFP) which is a marker protein for fetal hepatocytes (marker protein for hepatic stem/progenitor cells and hepatoblasts, which is not expressed in mature hepatocytes), and 324 ng/mL of AFP was observed in No. 473. The cell pellets were lysed in 1 mL/well of a QIAzol reagent (refer to Table 8B).

As described above, hepatic differentiation was induced by suspension (three-dimensional) culture in the presence of the TGF-β inhibitor to effectively prepare human induced hepatic progenitor cells or human hepatocytes from human induced hepatic stem cells.

### Example 7

### Induction of Hepatic Differentiation in the Presence of Oncostatin M and Dexamethasone

In a 10 cm-diameter culture dish coated with Matrigel (at an amount of 15 μL Matrigel/6 mL PBS/dish), the human induced hepatic stem cells AFB 1-1 (No. 451; about 50% confluence/dish), which were cocultured with feeder cells (1.5×10⁶ mouse embryonic fibroblasts (MEF)/dish) using the human ES/iPS cell medium (mTeSR1; STEMCELL Technologies) (supplemented with 100 ng/mL bFGF) and washed with PBS (−), were dissociated from the culture dish with a 0.25% trypsin/1 mM EDTA solution and suspended in the human ES/iPS cell medium (ReproStem; ReproCELL), then the suspension was subjected to centrifugal washing (at 1,000 rpm for 5 minutes). The human induced hepatic stem cells were suspended in ReproStem (bFGF-free)/Y-27632 (5 μM) supplemented with 10 ng/mL oncostatin M (OsM) and 0.1 μM dexamethasone (DEX), and were then seeded (at a density of about 8×10⁴ cells/5 mL medium/well) in a low-attachment 6-well culture plate (Corning; Cat. No. 3471) to culture the cells without feeder cells. In the present invention, the resultant cell sample is called “No. 474” (refer to Table 7B).

Six days after the seeding, the cells were recovered centrifugally and then suspended in 2 mL of a fresh ReproStem (bFGF-free)/Y-27632 (5 μM) supplemented with 10 ng/mL oncostatin M (OsM) and 0.1 μM dexamethasone (DEX), to continue hepatic differentiation culture in the above culture plate. On the following day, the centrifuged culture supernatant was subjected to measurement (SRL Inc.) for α-fetoprotein (AFP) which is a marker protein for fetal hepatocytes (marker protein for hepatic stem/progenitor cells and hepatoblasts, which is not expressed in mature hepatocytes), and 341 ng/mL of AFP was observed in No. 474. The cell pellets were lysed in 1 mL/well of a QIAzol reagent (refer to Table 8B).

As described above, hepatic differentiation was induced by suspension (three-dimensional) culture in the presence of oncostatin M and dexamethasone to effectively prepare human induced hepatic progenitor cells or human hepatocytes from human induced hepatic stem cells.

### Example 8

### Induction of Hepatic Differentiation in the Presence of Oncostatin M, Dexamethasone, and the TGF-β Inhibitor

In a 10 cm-diameter culture dish coated with Matrigel (at an amount of 15 μL Matrigel/6 mL PBS/dish), the human induced hepatic stem cells AFB 1-1 (No. 451; about 50% confluence/dish), which were cocultured with feeder cells (1.5×10⁶ mouse embryonic fibroblasts (MEF)/dish) using the human ES/iPS cell medium (mTeSR1; STEMCELL Technologies) (supplemented with 100 ng/mL bFGF) and washed with PBS (−), were dissociated from the culture dish with a 0.25% trypsin/1 mM EDTA solution and suspended in the human ES/iPS cell medium (ReproStem; ReproCELL), then the suspension was subjected to centrifugal washing (at 1,000 rpm for 5 minutes). The human induced hepatic stem cells were suspended in ReproStem (bFGF-free)/Y-27632 (5 μM) supplemented with 10 ng/mL oncostatin M (OsM), 0.1 μM dexamethasone (DEX), and 0.1 μM TGF-β inhibitor (A-83-01), and were then seeded (at a density of about 8×10⁴ cells/5 mL medium/well) in a low-attachment 6-well culture plate (Corning; Cat. No. 3471) to culture the cells without feeder cells. In the present invention, the resultant cell sample is called “No. 475” (refer to Table 7B).

Six days after the seeding, the cells were recovered centrifugally and then suspended in 2 mL of a fresh Y-27632 (5 μM)/ReproStem (bFGF-free) supplemented with 10 ng/mL oncostatin M (OsM), 0.1 μM dexamethasone (DEX), and 0.1 μM TGF-β inhibitor (A-83-01), to continue hepatic differentiation culture in the above culture plate. On the following day, the centrifuged culture supernatant was subjected to measurement (SRL Inc.) for α-fetoprotein (AFP) which is a marker protein for fetal hepatocytes (marker protein for hepatic stem/progenitor cells and hepatoblasts, which is not expressed in mature hepatocytes), and 262 ng/mL of AFP was observed in No. 475. The cell pellets were lysed in 1 mL/well of a QIAzol reagent (refer to Table 8B).

As described above, hepatic differentiation was induced by suspension (three-dimensional) culture in the presence of oncostatin M, dexamethasone, and the TGF-β inhibitor to effectively prepare human induced hepatic progenitor cells or human hepatocytes from human induced hepatic stem cells.

### Example 9

### Induction of Hepatic Differentiation in the Presence of Oncostatin M, Dexamethasone, the TGF-β Inhibitor, and Dimethylsulfoxide

In a 10 cm-diameter culture dish coated with Matrigel (at an amount of 15 μL Matrigel/6 mL PBS/dish), the human induced hepatic stem cells AFB 1-1 (No. 451; about 50% confluence/dish), which were cocultured with feeder cells (1.5×10⁶ mouse embryonic fibroblasts (MEF)/dish) using the human ES/iPS cell medium (mTeSR1; STEMCELL Technologies) (supplemented with 100 ng/mL bFGF) and washed with PBS (−), were dissociated from the culture dish with a 0.25% trypsin/1 mM EDTA solution and suspended in the human ES/iPS cell medium (ReproStem; ReproCELL), then the suspension was subjected to centrifugal washing (at 1,000 rpm for 5 minutes). The human induced hepatic stem cells were suspended in Y-27632 (5 μM)/ReproStem (bFGF-free) supplemented with 10 ng/mL oncostatin M (OsM), 0.1 μM dexamethasone (DEX), 0.1 μM TGF-β inhibitor (A-83-01), and 0.1% DMSO, and were then seeded (at a density of about 8×10⁴ cells/5 mL medium/well) in a low-attachment 6-well culture plate (Corning; Cat. No. 3471) to culture the cells without feeder cells. In the present invention, the resultant cell sample is called “No. 476” (refer to Table 7B).

Six days after the seeding, the cells were recovered centrifugally and then suspended in 2 mL of ReproStem (bFGF-free)/Y-27632 (5 μM) supplemented with 10 ng/mL oncostatin M (OsM), 0.1 μM dexamethasone (DEX), 0.1 μM TGF-β inhibitor (A-83-01), and 0.1% DMSO, to continue hepatic differentiation culture in the above culture plate. On the following day, the centrifuged culture supernatant was subjected to measurement (SRL Inc.) for α-fetoprotein (AFP) which is a marker protein for fetal hepatocytes (marker protein for hepatic stem/progenitor cells and hepatoblasts, which is not expressed in mature hepatocytes), and 417 ng/mL of AFP was observed in No. 476. The cell pellets were lysed in 1 mL/well of a QIAzol reagent (refer to Table 8B).

As described above, hepatic differentiation was induced by suspension (three-dimensional) culture in the presence of oncostatin M, dexamethasone, the TGF-β inhibitor, and dimethylsulfoxide to effectively prepare human induced hepatic progenitor cells or human hepatocytes from human induced hepatic stem cells.

### Example 10

### Induction of Hepatic Differentiation by Suspension (Three-Dimensional) Culture in the Absence of bFGF and in the Presence of Oncostatin M, Dexamethasone, the TGF-β Inhibitor, and Dimethylsulfoxide

In a 10 cm-diameter culture dish coated with Matrigel (at an amount of 15 μL Matrigel/6 mL PBS/dish), the human induced hepatic stem cells AFB 1-1 (No. 451; about 50% confluence/dish), which were cocultured with feeder cells (1.5×10⁶ mouse embryonic fibroblasts (MEF)/dish) using the human ES/iPS cell medium (mTeSR1; STEMCELL Technologies) (supplemented with 100 ng/mL bFGF) and washed with PBS (−), were dissociated from the culture dish with a 0.25% trypsin/1 mM EDTA solution and suspended in the human ES/iPS cell medium (ReproStem; ReproCELL), then the suspension was subjected to centrifugal washing (at 1,000 rpm for 5 minutes). The human induced hepatic stem cells were suspended in ReproStem (bFGF-free)/Y-27632 (5 μM) supplemented with 10 ng/mL oncostatin M (OsM), 0.1 μM dexamethasone (DEX), 0.1 μM TGF-β inhibitor (A-83-01), and 1% DMSO, and were then seeded (at a density of about 8×10⁴ cells/5 mL medium/well) in a low-attachment 6-well culture plate (Corning; Cat. No. 3471) to culture the cells without feeder cells. In the present invention, the resultant cell sample is called “No. 477” (refer to Table 7B).

Six days after the seeding, the cells were recovered centrifugally and then suspended in 2 mL of a fresh ReproStem (bFGF-free)/Y-27632 (5 μM) supplemented with 10 ng/mL oncostatin M (OsM), 0.1 μM dexamethasone (DEX), 0.1 μM TGF-β inhibitor (A-83-01), and 1% DMSO, to continue hepatic differentiation culture in the above culture plate. On the following day, the centrifuged culture supernatant was subjected to measurement (SRL Inc.) for α-fetoprotein (AFP) which is a marker protein for fetal hepatocytes (marker protein for hepatic stem/progenitor cells and hepatoblasts, which is not expressed in mature hepatocytes), and 427 ng/mL of AFP was observed in No. 477. The cell pellets were lysed in 1 mL/well of a QIAzol reagent (refer to Table 8B).

As described above, hepatic differentiation was induced by suspension (three-dimensional) culture in the absence of bFGF and in the presence of oncostatin M, dexamethasone, the TGF-β inhibitor, and dimethylsulfoxide to effectively prepare human induced hepatic progenitor cells or human hepatocytes from human induced hepatic stem cells.

TABLE 7A
Culture conditions (1)

| | Reference | Example 1 | Example 2 | Example 3 | Example 4 |
|---|---|---|---|---|---|
| Cell No. | No. 377 | No. 390 | No. 391 | No. 393 | No. 394 |
| Medium | mTeSR1 | mTeSR1 | aFGF/ | A-83-01/ | A-83-01/aFGF/ |
| | | | ReproStem | mTeSR1 | ReproStem |
| bFGF (ng/mL) | 100 | 100 | 0 | 100 | 0 |
| Feeder cells | Present | Absent | Absent | Absent | Absent |
| Matrigel | Present | Present | Present | Present | Present |

TABLE 7B
Culture conditions (2)

| Reference | Example 5 | Example 6 | Example 7 | Example 8 | Example 9 | Example 10 |
|---|---|---|---|---|---|---|
| No. 451 | No. 472 | No. 473 | No. 474 | No. 475 | No. 476 | No. 477 |
| ReproStem | ReproStem | ReproStem/ | OsM/DEX/ | OsM/DEX/ | OsM/DEX/ | OsM/DEX/ |
| | | A-83-01 | ReproStem | A-83-01/ | A-83-01/ | A-83-01/ |
| | | | | ReproStem | 0.1% DMSO/ | 1% DMSO/ |
| | | | | | ReproStem | ReproStem |
| 10 | 0 | 0 | 0 | 0 | 0 | 0 |
| Present | Absent | Absent | Absent | Absent | Absent | Absent |
| Present | Absent | Absent | Absent | Absent | Absent | Absent |

TABLE 8A
Summary of the results of Examples (1)

| | No. 390 | No. 391 | No. 393 | No. 394 |
|---|---|---|---|---|
| α- | 163 | 3,300 | 3,120 | 14,400 |
| fetoprotein | ng/mL | ng/mL | ng/mL | ng/mL |
| (AFP) | | | | |

Embryonic stem cell markers (as compared with
the respective marker expression levels in No.
377 being taken as 1)

| | |
|---|---|
| OCT3/4 (POU5F1) | 0.07 |
| SOX2 | 0.08 |
| NANOG | 0.01 |

Endoderm markers (as compared with the respective
marker expression levels in No. 377 being taken as 1)

| | |
|---|---|
| SOX17 | 0.03 |
| FOXA2 | 0.19 |
| GATA4 | 0.20 |

Hepatocyte transcription factors (as compared with
the respective marker expression levels in No.
377 being taken as 1)

| | |
|---|---|
| HNF1A | 0.88 |
| HNF4A | 0.39 |

Hepatic stem/progenitor cell markers (as compared with
the respective marker expression levels in No.
377 being taken as 1)

| | | | | |
|---|---|---|---|---|
| DLK1 | 264 | 786 | 404 | 804 |
| AFP | 126 | 3,420 | 1,791 | 12,812 |

Hepatocyte markers (as compared with the respective
marker expression levels in No. 377 being taken as 1)

| | | | | |
|---|---|---|---|---|
| ALB | 19 | 3,172 | 1,925 | 45,698 |
| AAT | 14 | 220 | 240 | 3,812 |
| TTR | 675 | 3,910 | 2,871 | 9,113 |
| FGG | | | | 10,138 |
| AHSG | | | | 14,079 |
| FABP1 | | | | 3,034 |
| RBP4 | | | | 4,326 |
| TF | | | | 9,126 |
| APOA4 | | | | 966 |

Other markers (as compared with the respective
marker expression levels in No. 377 being taken as 1)

| | |
|---|---|
| KRT7 | 37.5 |
| HGF | 11 |

TABLE 8B
Summary of the results of Examples (2)

| | No. 472 | No. 473 | No. 474 | No. 475 | No. 476 | No. 477 |
|---|---|---|---|---|---|---|
| α-fetoprotein | 349 ng/mL | 324 ng/mL | 341 ng/mL | 262 ng/mL | 417 ng/mL | 427 ng/mL |
| (AFP) | | | | | | |

### Example 11

### Induction of Hepatic Differentiation and Differentiation into Induced Hepatic Progenitor Cells in the Presence of a TGF-β Signaling Inhibitor (2)

In a 10 cm-diameter culture dish coated with Matrigel (at an amount of 60 μL Matrigel/6 mL PBS/dish for about one hour), the human induced hepatic stem cells NGC1-1 (No. 1133 (passage 45); about 50-80% confluence/dish), which were cocultured with feeder cells (about 1.5×10⁶ mouse embryonic fibroblasts (MEF)/60 cm² dish) using the human ES/iPS cell medium (mTeSR1; STEMCELL Technologies) and washed with PBS (−), were dissociated from the culture dish with a 0.25% trypsin/1 mM EDTA solution and suspended in the human ES/iPS cell medium (ReproStem; ReproCELL), then 1.2×10⁶ cells were subjected to centrifugal washing (at 1,000 rpm for 5 minutes).

The human induced hepatic stem cells were suspended in the medium ReproStem (supplemented with 10 ng/mL aFGF)/Y-27632 (10 μM) and then seeded (at a density of about 2×10⁵ cells/1 mL medium/well) in a 6-well plate coated with Matrigel (at an amount of 10 μL Matrigel/1 mL PBS/well for about one hour). After about three hours, the medium was replaced with 2 mL of ReproStem (supplemented with 10 ng/mL aFGF) containing 0.5 μM of one of the inhibitors listed below, and the human induced hepatic stem cells were subjected to culture for differentiation into human induced hepatic progenitor cells without feeder cells. The resultant cell samples are respectively referred to as “Nos. 1141-1145”.

No. 1140 was cultured in the absence of any inhibitor. Nos. 1141-1145 were cultured in the presence of the following inhibitors, respectively.
No. 1141: A-83-01 (TOCRIS; Cat. No. 2939)
No. 1142: ALK5 Inhibitor I ([3-(Pyridin-2-yl)-4-(4-quinonyl)]-1H-pyrazole; MERCK Calbiochem; Cat. No. 616451)
No. 1143: TGF-β RI Kinase Inhibitor II (2-(3-(6-Methylpyridin-2-yl)-1H-pyrazol-4-yl)-1,5-naphthyridine; MERCK Calbiochem; Cat. No. 616452)
No. 1144: SB431542 (4-[4-(1,3-Benzodioxol-5-yl)-5-pyridin-2-yl-1H-imidazol-2-yl]benzamide, Dihydrate; Cayman; Cat. No. 13031)
No. 1145: LY-364947 (4-[3-(2-pyridinyl)-1H-pyrazol-4-yl]-quinoline; Cayman; Cat. No. 13341).

Three, five and six days after the seeding, each medium was replaced with a fresh medium of the same composition containing the individual inhibitor, and the cells were subjected to differentiation culture. Seven days after the seeding, the cells were lysed in 1 mL/well of a QIAzol reagent to prepare the total RNA from the cell lysate using the miRNeasy Mini Kit (Qiagen). The total RNA was subjected to quantitative RT-PCR using the iScript Advanced cDNA synthesis kit, the SsoAdvanced SYBR Green Supremix (2 mL), and the CFX96 Real-Time System C1000 Thermal Cycler, all manufactured by Bio-Rad. The quantified genes were albumin (ALB), α1-antitrypsin (AAT), transthyretin (TTR), and α-fetoprotein (AFP).

On the basis of the results of the quantitative RT-PCR, in the human induced hepatic progenitor cell samples (Nos. 1140, 1141, 1142, 1143, 1144 and 1145):

the ALB expression level increased by 24.11, 393.55, 163.71, 296.67, 94.46 and 114.78 times, respectively;
the AAT expression level increased by 3.00, 19.83, 13.45, 22.18, 12.15 and 14.36 times, respectively;
the TTR expression level increased by 128.22, 935.16, 966.14, 1,262.14, 614.17 and 482.45 times, respectively; and
the AFP expression level increased by 33.02, 655.37, 747.65, 720.03, 394.40 and 369.23 times, respectively,
as compared the respective marker expression levels in the human induced hepatic stem cells (No. 1133) being taken as 1.

As is evident from the above-noted results, the culture procedures in the presence of a TGF-β signaling inhibitor were suitable for effectively preparing human induced hepatic progenitor cells from human induced hepatic stem cells. These culture procedures were also considered to be suitable for preparing human hepatocytes from human induced hepatic stem cells or human induced hepatic progenitor cells.

TABLE 9
Culture conditions and results

| | Cell No. | | | | | | |
|---|---|---|---|---|---|---|---|
| | No. 1133 | No. 1140 | | | | | |
| | (Reference) | (Reference) | No. 1141 | No. 1142 | No. 1143 | No. 1144 | No. 1145 |
| Medium | mTeSR1 | ReproStem | A-83-01/ | 616451/ | 616452/ | SB431542/ | LY-364947/ |
| | | | ReproStem | ReproStem | ReproStem | ReproStem | ReproStem |
| bFGF | 100 | 0 | 0 | 0 | 0 | 0 | 0 |
| (ng/mL) | | | | | | | |
| Feeder cells | (+) | (−) | (−) | (−) | (−) | (−) | (−) |
| Matrigel | (+) | (+) | (+) | (+) | (+) | (+) | (+) |
| ALB | 1 | 24.11 | 393.55 | 163.71 | 296.67 | 94.46 | 114.78 |
| expression | | | | | | | |
| ratio | | | | | | | |
| AAT | 1 | 3.00 | 19.83 | 13.45 | 22.18 | 12.15 | 14.36 |
| expression | | | | | | | |
| ratio | | | | | | | |
| TTR | 1 | 128.22 | 935.16 | 966.14 | 1,262.14 | 614.17 | 482.45 |
| expression | | | | | | | |
| ratio | | | | | | | |
| AFP | 1 | 33.02 | 655.37 | 747.65 | 720.03 | 394.40 | 369.23 |
| expression | | | | | | | |
| ratio | | | | | | | |

### Example 12

### Induction of Hepatic Differentiation and Differentiation into Induced Hepatic Progenitor Cells in the Presence of a TGF-β Signaling Inhibitor (3)

In a 10 cm-diameter culture dish coated with Matrigel (at an amount of 60 μL Matrigel/6 mL PBS/dish for about one hour), the human induced hepatic stem cells AFB1-1 No. 1543 (passage 36) which had been cryopreserved in liquid nitrogen were cocultured with feeder cells (about 1.5×10⁶ mouse embryonic fibroblasts (MEF)/60 cm² dish) using the human ES/iPS cell medium (mTeSR1; STEMCELL Technologies).

After reaching 50-70% confluence, the cells were washed with PBS (−), dissociated from the culture dish with a 0.25% trypsin/1 mM EDTA solution and then suspended in the human ES/iPS cell medium (ReproStem; ReproCELL), then 1.2×10⁶ cells were subjected to centrifugal washing (at 1,000 rpm for 5 minutes). The human induced hepatic stem cells were suspended in the medium ReproStem (bFGF-free)/Y-27632 (10 μM) and then seeded (at a density of about 2×10⁵ cells/1 mL medium/well) in a 6-well plate coated with Matrigel (at an amount of 10 μL Matrigel/1 mL PBS/well for about one hour).

After about six hours, the medium was replaced with 2 mL of ReproStem (bFGF-free) supplemented with 0.5 μM of one of the inhibitors listed below, and the human induced hepatic stem cells were subjected to culture for differentiation into human induced hepatic progenitor cells without feeder cells. The resultant cell samples are respectively referred to as “Nos. 1545-1549”. No. 1544 was cultured in the absence of any inhibitor. Nos. 1545-1549 were cultured in the presence of the following inhibitors, respectively.

No. 1545: A-83-01 (TOCRIS; Cat. No. 2939)
No. 1546: SB-505124 (2-(5-benzo[1,3]dioxol-5-yl-2-tert-butyl-3H-imidazol-4-yl)-6-methylpyridine hydrochloride; SIGMA; Cat No. 54696)
No. 1547: TGF-β RI Inhibitor III (2-(5-Benzo[1,3]dioxol-4-yl-2-tert-butyl-1H-imidazol-4-yl)-6-methylpyridine, HCl; MERCK Calbiochem; Cat. No. 616453)
No. 1548: SD-208, TGF-β RI Inhibitor V (2-(5-Chloro-2-fluorophenyl)pteridin-4-yl)pyridin-4-ylamine; MERCK Calbiochem; Cat. No. 616456)
No. 1549: TGF-β RI Kinase Inhibitor VIII (6-(2-tert-Butyl-5-(6-methyl-pyridin-2-yl)-1H-imidazol-4-yl)-quinoxaline; CALBIO; Cat. No. 616459)

After the seeding, each medium was replaced every two or three days with a fresh medium of the same composition containing the individual inhibitor, and the cells were subjected to differentiation culture. Thirteen days after the seeding, the cells were lysed in 1 mL/well of a QIAzol reagent to prepare the total RNA from the cell lysate using the miRNeasy Mini Kit (Qiagen). The total RNA was subjected to quantitative RT-PCR using the iScript Advanced cDNA synthesis kit, the SsoAdvanced SYBR Green Supremix (2 mL), and the CFX96 Real-Time System C1000 Thermal Cycler, all manufactured by Bio-Rad. The quantified genes were ALB, AAT, TTR, AFP, cytokeratin 7 (KRT7), cytokeratin 19 (KRT19), and DLK1 (Delta-like 1 homolog).

On the basis of the results of the quantitative RT-PCR, the respective marker expression levels in the human induced hepatic progenitor cell samples (Nos. 1545, 1546, 1547, 1548, 1549) were compared to determine the following Ct values:

the ALB expression of Nos. 1545-1549 were 20.95, 23.2, 25.55, 21.35, and 24.67, respectively;
the AAT expression of Nos. 1545-1549 were 23.57, 23.56, 24.09, 23.54, and 23.54, respectively;
the TTR expression of Nos. 1545-1549 were 16.96, 17.24, 18.13, 17.4, and 17.24, respectively;
the AFP expression of Nos. 1545-1549 were 17.21, 18.61, 20.24, 17.48, and 19.25, respectively;
the KRT7 expression of Nos. 1545-1549 were 20.51, 19.8, 19.45, 20.05, and 19.89, respectively;
the KRT19 expression of Nos. 1545-1549 were 22.05, 20.33, 20.29, 20.45, and 20.36, respectively;
the DLK1 expression of Nos. 1545-1549 were 18.15, 18.77, 18.74, 19.1, and 18.66, respectively; and
the GAPDH expression of Nos. 1545-1549 were 14.22, 13.25, 13.76, 13.72, and 14.24, respectively.
As shown above, the hepatocyte markers, the hepatic progenitor cell markers, and the biliary duct epithelial cell markers were detected. Thus, hepatic differentiation was induced in the presence of a TGF-β signaling inhibitor to achieve differentiation into induced hepatic progenitor cells.

Further, as compared with the marker expression level in the human induced hepatic stem cells (No. 1543) being taken as 1, the expression level of KRT7 (hepatic progenitor cell marker or biliary duct epithelial cell marker) in the human induced hepatic progenitor cell samples (Nos. 1544, 1545, 1546, 1547, 1548, 1549) significantly increased by 655, 3291, 2768, 4761, 3186, and 4905 times, respectively.

As is evident from the above-noted results, the culture procedures in the presence of a TGF-β signaling inhibitor were suitable for effectively differentiating human induced hepatic progenitor cells from human induced hepatic stem cells. These culture procedures were also considered to be suitable for differentiating human hepatocytes from human induced hepatic stem cells or human induced hepatic progenitor cells (refer to Table 10).

TABLE 10
Culture conditions and results

| | Cell No. | | | | | | |
|---|---|---|---|---|---|---|---|
| | No. 1543 | No. 1544 | | | | | |
| | (Reference) | (Reference) | No. 1545 | No. 1546 | No. 1547 | No. 1548 | No. 1549 |
| Medium | mTeSR1 | ReproStem | A-83-01/ | SB-505124/ | 616453/ | 616456/ | 616459/ |
| | | | ReproStem | ReproStem | ReproStem | ReproStem | ReproStem |
| bFGF | 100 | 0 | 0 | 0 | 0 | 0 | 0 |
| (ng/mL) | | | | | | | |
| Feeder cells | (+) | (−) | (−) | (−) | (−) | (−) | (−) |
| Matrigel | (+) | (+) | (+) | (+) | (+) | (+) | (+) |
| ALB | — | — | 20.95 | 23.2 | 25.55 | 21.35 | 24.67 |
| expression | | | | | | | |
| Ct value | | | | | | | |
| AAT | — | — | 23.57 | 23.56 | 24.09 | 23.54 | 23.54 |
| expression | | | | | | | |
| Ct value | | | | | | | |
| TTR | — | — | 16.96 | 17.24 | 18.13 | 17.4 | 17.24 |
| expression | | | | | | | |
| Ct value | | | | | | | |
| AFP | — | — | 17.21 | 18.61 | 20.24 | 17.48 | 19.25 |
| expression | | | | | | | |
| Ct value | | | | | | | |
| KRT7 | — | — | 20.51 | 19.8 | 19.45 | 20.05 | 19.89 |
| expression | | | | | | | |
| Ct value | | | | | | | |
| KRT19 | — | — | 22.05 | 20.33 | 20.29 | 20.45 | 20.36 |
| expression | | | | | | | |
| Ct value | | | | | | | |
| DLK1 | — | — | 18.15 | 18.77 | 18.74 | 19.1 | 18.66 |
| expression | | | | | | | |
| Ct value | | | | | | | |
| GAPDH | — | — | 14.22 | 13.25 | 13.76 | 13.72 | 14.24 |
| expression | | | | | | | |
| Ct value | | | | | | | |
| KRT7 | 1 | 655 | 3,291 | 2768 | 4761 | 3186 | 4905 |
| expression | | | | | | | |
| ratio | | | | | | | |

### Example 13

### Induction of Hepatic Differentiation, and Preparation of Induced Hepatic Progenitor Cells, in the Absence of bFGF/aFGF

In a 10 cm-diameter culture dish coated with Matrigel (at an amount of 60 μL Matrigel/6 mL PBS/dish for about one hour), the human induced hepatic stem cells AFB1-1 (No. 806 (passage 42); about 50-80% confluence/dish), which were cocultured with feeder cells (about 1.5×10⁶ mouse embryonic fibroblasts (MEF)/60 cm² dish) using the human ES/iPS cell medium (ReproStem; ReproCELL) supplemented with 10 ng/mL bFGF and washed with PBS (−), were dissociated from the culture dish with a 0.25% trypsin/1 mM EDTA solution and suspended in the human ES/iPS cell medium (ReproStem; ReproCELL), then a tenth of the suspension was subjected to centrifugal washing (at 1,000 rpm for 5 minutes).

The human induced hepatic stem cells were suspended in the human ES/iPS cell medium (mTeSR1; STEMCELL Technologies)/Y-27632 (10 μM) and then seeded for coculture on feeder cells (about 1.5×10⁶ mouse embryonic fibroblasts (MEF)/60 cm² dish) seeded in a 10 cm-diameter culture dish coated with Matrigel (at an amount of 10 μL Matrigel/1 mL PBS/well for about one hour).

The medium was replaced everyday with a fresh human ES/iPS cell medium (mTeSR1) to continue culture until reaching 50-80% confluence per dish. The human induced hepatic stem cells AFB1-1 (No. 834 (passage 43)) were washed with PBS (−), and were then dissociated from the culture dish with a 0.25% trypsin/1 mM EDTA solution and suspended in the human ES/iPS cell medium (ReproStem; ReproCELL), then 1.2×10⁶ cells were subjected to centrifugal washing (at 1,000 rpm for 5 minutes). The human induced hepatic stem cells were suspended in the medium ReproStem (bFGF-free)/Y-27632 (10 μM) and then seeded (at a density of about 2×10⁵ cells/1 mL medium/well) in a 6-well plate coated with Matrigel (at an amount of 10 μL Matrigel/1 mL PBS/well for about one hour). After about three hours, the medium was replaced with 2 mL of ReproStem (bFGF-free) supplemented with 0.5 μM A-83-01, and the human induced hepatic stem cells were subjected to culture for differentiation into human induced hepatic progenitor cells without feeder cells. The resultant cell sample is referred to as “No. 835”.

After the seeding, the medium was replaced every two or three days with a fresh medium of the same composition containing 0.5 μM A-83-01, and the cells were subjected to differentiation culture. Six and thirteen days after the seeding, the culture supernatant was subjected to measurement (SRL Inc. (CRO)) for AFP, and 6,430 ng/mL and 30,900 ng/mL of AFPs were observed in No. 835 on the respective days.

Thirteen days after the seeding, the cells were lysed in 1 mL/well of a QIAzol reagent to prepare the total RNA from the cell lysate using the miRNeasy Mini Kit (Qiagen). The total RNA was subjected to quantitative RT-PCR using the iScript Advanced cDNA synthesis kit, the SsoAdvanced SYBR Green Supremix (2 mL), and the CFX96 Real-Time System C1000 Thermal Cycler, all manufactured by Bio-Rad. The quantified genes were ALB, AAT, TTR, and AFP.

According to the results of the quantitative RT-PCR, the expression level of ALB in the human induced hepatic progenitor cell sample (No. 835) increased by 11,300 times as compared with the marker expression level in the human induced hepatic stem cells (No. 806) being taken as 1. The expression levels of AAT, TTR and AFP in No. 835 also increased by 98.1, 312.2 and 145.0 times, respectively, as compared with those levels in No. 806 in the same way.

As is evident from the above-noted results, the culture procedure in the presence of A-83-01 was suitable for effectively differentiating human induced hepatic progenitor cells from human induced hepatic stem cells. This culture procedure was also considered to be suitable for differentiating human hepatocytes from human induced hepatic stem cells or human induced hepatic progenitor cells (refer to Table 11).

TABLE 11
Culture conditions and results

| | Cell No. | | | |
|---|---|---|---|---|
| | No. 806 | | | |
| | (Reference) | No. 834 | No. 835 | |
| | Medium | | | |
| | | | A-83-01/ReproStem | |
| | | | 6 days after | 13 days after |
| | ReproStem | mTeSR1 | the seeding | the seeding |
| bFGF | 10 | 100 | 0 | 0 |
| (ng/mL) | | | | |
| Feeder | (+) | (+) | (−) | (−) |
| cells | | | | |
| Matrigel | (+) | (+) | (+) | (+) |
| AFP yield | — | — | 6,430 ng/mL | 30,900 ng/mL |
| ALB | 1 | — | — | 11,300 |
| expression | | | | |
| ratio | | | | |
| AAT | 1 | — | — | 98.1 |
| expression | | | | |
| ratio | | | | |
| TTR | 1 | — | — | 312.2 |
| expression | | | | |
| ratio | | | | |
| AFP | 1 | — | — | 145.0 |
| expression | | | | |
| ratio | | | | |

### Example 14

### Induction of Hepatic Differentiation, and Preparation of Induced Hepatic Progenitor Cells, in the Presence of Steroid Hormones

In a 10 cm-diameter culture dish coated with Matrigel (at an amount of 60 μL Matrigel/6 mL PBS/dish for about one hour), the human induced hepatic stem cells NGC1-1 (No. 946 (passage 37); about 50-80% confluence/dish), which were cocultured with feeder cells (about 1.5×10⁶ mouse embryonic fibroblasts (MEF)/60 cm² dish) using the human ES/iPS cell medium (ReproStem; ReproCELL) supplemented with 10 ng/mL bFGF and washed with PBS (−), were dissociated from the culture dish with a 0.25% trypsin/1 mM EDTA solution and suspended in the human ES/iPS cell medium (ReproStem; ReproCELL), then a tenth of the suspension to centrifugal washing (at 1,000 rpm for 5 minutes).

The human induced hepatic stem cells were suspended in the human ES/iPS cell medium (mTeSR1; STEMCELL Technologies)/Y-27632 (10 μM) and then seeded for coculture on feeder cells (about 1.5×10⁶ mouse embryonic fibroblasts (MEF)/60 cm² dish) seeded in a 10 cm-diameter culture dish coated with Matrigel (at an amount of 10 μL Matrigel/1 mL PBS/well for about one hour).

The medium was replaced everyday with a fresh human ES/iPS cell medium (mTeSR1) to continue culture until reaching 50-80% confluence per dish. The human induced hepatic stem cells NGC1-1 (No. 947 (passage 38)) were washed with PBS (−), and were then dissociated from the culture dish with a 0.25% trypsin/1 mM EDTA solution and suspended in the human ES/iPS cell medium (ReproStem; ReproCELL), then 1.2×10⁶ cells were subjected to centrifugal washing (at 1,000 rpm for 5 minutes). The human induced hepatic stem cells were suspended in the medium ReproStem (bFGF-free)/Y-27632 (10 μM) and then seeded (at a density of about 2×10⁵ cells/1 mL medium/well) in a 6-well plate coated with Matrigel (at an amount of 10 μL Matrigel/1 mL PBS/well for about one hour). After about three hours, the medium was replaced with 2 mL of ReproStem (bFGF-free) supplemented with not only 0.5 μM A-83-01 but also 0.1 μM estrone, 0.1 μM estradiol, 0.1 μM estriol, 10 μM progesterone, 0.1 μM cortisone, 0.1 μM aldosterone, 0.01 nM triiodothyronine, 0.01 nM thyroxine, 0.1 μM testosterone, and 0.1 μM dehydroepiandrosterone, and the human induced hepatic stem cells were subjected to culture for differentiation into human induced hepatic progenitor cells without feeder cells. The resultant cell sample is referred to as “No. 949”.

After the seeding, the medium was replaced every two or three days with a fresh medium of the same composition, and the cells were subjected to differentiation culture. Thirteen days after the seeding, the cells were lysed in 1 mL/well of a QIAzol reagent to prepare the total RNA from the cell lysate using the miRNeasy Mini Kit (Qiagen). The total RNA was subjected to quantitative RT-PCR using the iScript Advanced cDNA synthesis kit, the SsoAdvanced SYBR Green Supremix (2 mL), and the CFX96 Real-Time System C1000 Thermal Cycler, all manufactured by Bio-Rad. The quantified genes were ALB, AAT, TTR, AFP and CYP1A2.

According to the results of the quantitative RT-PCR, the expression level of ALB in the human induced hepatic progenitor cell sample (No. 949) increased by 7,212 times as compared with the marker expression level in the human induced hepatic stem cells (No. 946) being taken as 1. The expression levels of AAT, TTR, AFP and CYP1A2 in No. 949 also increased by 34, 725, 86 and 12.280 times, respectively, as compared with those levels in No. 946 in the same way.

As is evident from the above-noted results, the culture procedure in the presence of steroid hormones was suitable for effectively differentiating human induced hepatic progenitor cells from human induced hepatic stem cells. This culture procedure was also considered to be suitable for differentiating human hepatocytes from human induced hepatic stem cells or human induced hepatic progenitor cells (refer to Table 12).

### Example 15

### Induction of Hepatic Differentiation, and Preparation of Induced Hepatic Progenitor Cells, in the Presence of Bile Acids, Fatty Acid, and Cholesterol

In a 10 cm-diameter culture dish coated with Matrigel (at an amount of 60 μL Matrigel/6 mL PBS/dish for about one hour), the human induced hepatic stem cells NGC1-1 (No. 946 (passage 37); about 50-80% confluence/dish), which were cocultured with feeder cells (about 1.5×10⁶ mouse embryonic fibroblasts (MEF)/60 cm² dish) using the human ES/iPS cell medium (ReproStem; ReproCELL) supplemented with 10 ng/mL bFGF and washed with PBS (−), were dissociated from the culture dish with a 0.25% trypsin/1 mM EDTA solution and suspended in the human ES/iPS cell medium (ReproStem; ReproCELL), then a tenth of the suspension was subjected to centrifugal washing (at 1,000 rpm for 5 minutes).

The human induced hepatic stem cells were suspended in the human ES/iPS cell medium (mTeSR1; STEMCELL Technologies) supplemented with Y-27632 (10 μM) and then seeded for coculture on feeder cells (about 1.5×10⁶ mouse embryonic fibroblasts (MEF)/60 cm² dish) seeded in a 10 cm-diameter culture dish coated with Matrigel (at an amount of 10 μL Matrigel/1 mL PBS/well for about one hour). The medium was replaced everyday with a fresh human ES/iPS cell medium (mTeSR1) to continue culture until reaching 50-80% confluence per dish. The human induced hepatic stem cells NGC1-1 (No. 947 (passage 38)) were washed with PBS (−), and were then dissociated from the culture dish with a 0.25% trypsin/1 mM EDTA solution and suspended in the human ES/iPS cell medium (ReproStem; ReproCELL), then 1.2×10⁶ cells were subjected to centrifugal washing (at 1,000 rpm for 5 minutes). The human induced hepatic stem cells were suspended in the medium ReproStem (bFGF-free)/Y-27632 (10 μM) and then seeded (at a density of about 2×10⁵ cells/1 mL medium/well) in a 6-well plate coated with Matrigel (at an amount of 10 μL Matrigel/1 mL PBS/well for about one hour). After about three hours, the medium was replaced with 2 mL of ReproStem (bFGF-free) supplemented with not only 0.5 μM A-83-01 but also 5 μM cholic acid, 5 μM chenodeoxycholic acid, 250× fatty acid concentrate (Invitrogen; Cat. No. 11905-031) × 1/250, and 250× cholesterol concentrate (Invitrogen; Cat. No. 12531-018) × 1/250, and the human induced hepatic stem cells were subjected to culture for differentiation into human induced hepatic progenitor cells without feeder cells. The resultant cell sample is referred to as “No. 951”.

After the seeding, the medium was replaced every two or three days with a fresh medium of the same composition, and the cells were subjected to differentiation culture. Thirteen days after the seeding, the cells were lysed in 1 mL/well of a QIAzol reagent to prepare the total RNA from the cell lysate using the miRNeasy Mini Kit (Qiagen). The total RNA was subjected to quantitative RT-PCR using the iScript Advanced cDNA synthesis kit, the SsoAdvanced SYBR Green Supremix (2 mL), and the CFX96 Real-Time System C1000 Thermal Cycler, all manufactured by Bio-Rad. The quantified genes were ALB, AAT, TTR, AFP and CYP1A2.

According to the results of the quantitative RT-PCR, the expression level of ALB in the human induced hepatic progenitor cell sample (No. 951) increased by 9,306 times as compared with the marker expression level in the human induced hepatic stem cells (No. 946) being taken as 1. The expression levels of AAT, TTR, AFP and CYP1A2 in No. 951 also increased by 144, 948, 220 and 7.235 times, respectively, as compared with those levels in No. 946 in the same way.

As is evident from the above-noted results, the culture procedure in the presence of bile acids, fatty acid, and cholesterol was suitable for effectively differentiating human induced hepatic progenitor cells from human induced hepatic stem cells. This culture procedure was also considered to be suitable for differentiating human hepatocytes from human induced hepatic stem cells or human induced hepatic progenitor cells.

TABLE 12
Culture conditions and results

| | Cell No. | | | |
|---|---|---|---|---|
| | No. 946 | No. 947 | | |
| | (Reference) | (Reference) | No. 949 | No. 951 |
| | Medium | | | |
| | | | A-83-01 + | A-83-01 + |
| | | | estrone, etc./ | cholic acid, etc./ |
| | ReproStem | mTeSR1 | ReproStem | ReproStem |
| bFGF | 10 | 100 | 0 | 0 |
| (ng/mL) | | | | |
| Feeder | (+) | (+) | (−) | (−) |
| cells | | | | |
| Matrigel | (+) | (+) | (+) | (+) |
| ALB | 1 | — | 7,212 | 9,306 |
| expression | | | | |
| ratio | | | | |
| AAT | 1 | — | 34 | 144 |
| expression | | | | |
| ratio | | | | |
| TTR | 1 | — | 725 | 948 |
| expression | | | | |
| ratio | | | | |
| AFP | 1 | — | 86 | 220 |
| expression | | | | |
| ratio | | | | |
| CYP1A2 | 1 | — | 12.280 | 7.235 |
| expression | | | | |
| ratio | | | | |

### Example 16

### Induction of Hepatic Differentiation, and Preparation of Induced Hepatic Progenitor Cells, in the Presence of Serum and Dexamethasone

In a 10 cm-diameter culture dish coated with Matrigel (at an amount of 60 μL Matrigel/6 mL PBS/dish for about one hour), the human induced hepatic stem cells AFB1-1, which were cocultured with feeder cells (about 1.5×10⁶ mouse embryonic fibroblasts (MEF)/60 cm² dish) using the human ES/iPS cell medium (ReproStem; ReproCELL) supplemented with 10 ng/mL bFGF and washed with PBS (−), were dissociated from the culture dish with a 0.25% trypsin/1 mM EDTA solution and suspended in the human ES/iPS cell medium (ReproStem; ReproCELL), then a tenth of the suspension was subjected to centrifugal washing (at 1,000 rpm for 5 minutes).

The human induced hepatic stem cells were suspended in the human ES/iPS cell medium (mTeSR1; STEMCELL Technologies)/Y-27632 (10 μM) and then seeded for coculture on feeder cells (about 1.5×10⁶ mouse embryonic fibroblasts (MEF)/60 cm² dish) seeded in a 10 cm-diameter culture dish coated with Matrigel (at an amount of 10 μL Matrigel/1 mL PBS/well for about one hour). The medium was replaced everyday with a fresh human ES/iPS cell medium (mTeSR1) to continue culture until reaching 50-80% confluence per dish.

The human induced hepatic stem cells AFB1-1 (No. 664 (passage 35)) were washed with PBS (−), and were then dissociated from the culture dish with a 0.25% trypsin/1 mM EDTA solution and suspended in the human ES/iPS cell medium (ReproStem; ReproCELL), then 1.2×10⁶ cells were subjected to centrifugal washing (at 1,000 rpm for 5 minutes). The human induced hepatic stem cells were suspended in the medium ReproStem (bFGF-free)/Y-27632 (10 μM) and then seeded (at a density of about 2×10⁵ cells/1 mL medium/well) in a 6-well plate coated with Matrigel (at an amount of 10 μL Matrigel/1 mL PBS/well for about one hour). After about three hours, the medium was replaced with 2 mL of DMEM/10% FBS containing 0.5 μM A-83-01, and the human induced hepatic stem cells were subjected to culture for differentiation into human induced hepatic progenitor cells without feeder cells.

After the seeding, the medium was replaced every two or three days with a fresh medium of the same composition containing 0.5 μM A-83-01, and the cells were subjected to differentiation culture. Six days after the seeding, the medium was changed to a 10% fetal bovine serum (FBS)-supplemented DMEM medium supplemented with not only 0.5 μM A-83-01 but also 0.1 μM (No. 683), 0.5 μM (No. 684) or 2 μM (No. 685) of dexamethasone (DEX), and replaced every two or three days with a fresh one. Fourteen days after the seeding, the cells were lysed in 1 mL/well of a QIAzol reagent to prepare the total RNA from the cell lysate using the miRNeasy Mini Kit (Qiagen). The total RNA was subjected to quantitative RT-PCR using the iScript Advanced cDNA synthesis kit, the SsoAdvanced SYBR Green Supremix (2 mL), and the CFX96 Real-Time System C1000 Thermal Cycler, all manufactured by Bio-Rad. The quantified genes were ALB, AAT, TTR, AFP and CYP3A4.

On the basis of the results of the quantitative RT-PCR, in the human induced hepatic progenitor cell samples (Nos. 683, 684, 685):

the ALB expression level increased by 11,284, 16,667 and 13,278 times, respectively;
the AAT expression level increased by 70.4, 90.9 and 78.3 times, respectively;
the TTR expression level increased by 59.3, 83.3 and 78.6 times, respectively; and
the AFP expression level increased by 7,178, 10,000 and 6931 times, respectively,
as compared with the respective marker expression levels in the human induced hepatic stem cells (No. 663) being taken as 1. And the CYP3A4 expression level in Nos. 683-685 increased by 1,003, 1,389 and 1,038 times as compared with the marker expression level in the human induced hepatic stem cells AFB1-1 (No. 664) being taken as 1.

As is evident from the above-noted results, the culture procedures in the presence of serum and dexamethasone (DEX) were suitable for effectively differentiating human induced hepatic progenitor cells from human induced hepatic stem cells. These culture procedures were also considered to be suitable for differentiating human hepatocytes from human induced hepatic progenitor cells.

TABLE 13
Culture conditions and results

| | Cell No. | | | | |
|---|---|---|---|---|---|
| | No. 663 | No. 664 | | | |
| | (Reference) | (Reference) | No. 683 | No. 684 | No. 685 |
| | Medium | | | | |
| | ReproStem | mTeSR1 | A-83-01/DMEM | A-83-01/DMEM | A-83-01/DMEM |
| bFGF | 10 | 100 | 0 | 0 | 0 |
| (ng/mL) | | | | | |
| Feeder | (+) | (+) | (−) | (−) | (−) |
| cells | | | | | |
| Matrigel | (+) | (+) | (+) | (+) | (+) |
| ALB | 1 | — | 11,284 | 16,667 | 13,278 |
| expression | | | | | |
| ratio | | | | | |
| AAT | 1 | — | 70.4 | 90.9 | 78.3 |
| expression | | | | | |
| ratio | | | | | |
| TTR | 1 | — | 59.3 | 83.3 | 78.6 |
| expression | | | | | |
| ratio | | | | | |
| AFP | 1 | — | 7,178 | 10,000 | 6931 |
| expression | | | | | |
| ratio | | | | | |
| CYP3A4 | — | 1 | 1,003 | 1,389 | 1,038 |
| expression | | | | | |
| ratio | | | | | |

### Example 17

### Induction of Hepatic Differentiation, and Preparation of Induced Hepatic Progenitor Cells, in the Presence of TGF-β Signaling Inhibitor and Dexamethasone

In a 10 cm-diameter culture dish coated with Matrigel (at an amount of 60 μL Matrigel/6 mL PBS/dish for about one hour), the human induced hepatic stem cells AFB1-1, which were cocultured with feeder cells (about 1.5×10⁶ mouse embryonic fibroblasts (MEF)/60 cm² dish) using the human ES/iPS cell medium (ReproStem; ReproCELL) supplemented with 10 ng/mL bFGF and washed with PBS (−), were dissociated from the culture dish with a 0.25% trypsin/1 mM EDTA solution and suspended in the human ES/iPS cell medium (ReproStem; ReproCELL), then a tenth of the suspension was subjected to centrifugal washing (at 1,000 rpm for 5 minutes).

The human induced hepatic stem cells were suspended in the human ES/iPS cell medium (mTeSR1; STEMCELL Technologies)/Y-27632 (10 μM) and then seeded for coculture on feeder cells (about 1.5×10⁶ mouse embryonic fibroblasts (MEF)/60 cm² dish) seeded in a 10 cm-diameter culture dish coated with Matrigel (at an amount of 10 μL Matrigel/1 mL PBS/well for about one hour). The medium was replaced everyday with a fresh human ES/iPS cell medium (mTeSR1) to continue culture until reaching 50-80% confluence per dish. The human induced hepatic stem cells AFB1-1 (No. 664 (passage 35)) were washed with PBS (−), and were then dissociated from the culture dish with a 0.25% trypsin/1 mM EDTA solution and suspended in the human ES/iPS cell medium (ReproStem; ReproCELL), then 1.2×10⁶ cells were subjected to centrifugal washing (at 1,000 rpm for 5 minutes). The human induced hepatic stem cells were suspended in the medium ReproStem (bFGF-free)/Y-27632 (10 μM) and then seeded (at a density of about 2×10⁵ cells/1 mL medium/well) in a 6-well plate coated with Matrigel (at an amount of 10 μL Matrigel/1 mL PBS/well for about one hour). After about three hours, the medium was replaced with 2 mL of ReproStem (bFGF-free) containing 0.5 μM A-83-01, and the human induced hepatic stem cells were subjected to culture for differentiation into human induced hepatic progenitor cells without feeder cells.

After the seeding, the medium was replaced every two or three days with a fresh medium of the same composition containing 0.5 μM A-83-01, and the cells were subjected to differentiation culture. Six days after the seeding, the medium was changed to a ReproStem (bFGF-free) medium supplemented with not only 0.5 μM A-83-01 but also 0.1 μM (No. 686), 0.5 μM (No. 687) or 2 μM (No. 688) of dexamethasone (DEX), and replaced every two or three days with a fresh one. Fourteen days after the seeding, the cells were lysed in 1 mL/well of a QIAzol reagent to prepare the total RNA from the cell lysate using the miRNeasy Mini Kit (Qiagen). The total RNA was subjected to quantitative RT-PCR using the iScript Advanced cDNA synthesis kit, the SsoAdvanced SYBR Green Supremix (2 mL), and the CFX96 Real-Time System C1000 Thermal Cycler, all manufactured by Bio-Rad. The quantified genes were ALB, AAT, TTR, AFP, CYP1A2, CYP2C9, and CYP3A4.

On the basis of the results of the quantitative RT-PCR, in the human induced hepatic progenitor cell samples (Nos. 686, 687, 688):

the ALB expression level increased by 3,706, 4,306 and 2,559 times, respectively;
the AAT expression level increased by 201, 224 and 129 times, respectively;
the TTR expression level increased by 156, 166 and 89 times, respectively; and
the AFP expression level increased by 4,414, 4,227 and 3,414 times, respectively,
as compared with the respective marker expression levels in the human induced hepatic stem cells (No. 663) being taken as 1. Also in Nos. 686-688:
the CYP1A2 expression level increased by 6.4, 4.9 and 10.8 times, respectively;
the CYP2C9 expression level increased by 9.0, 6.6 and 4.5 times, respectively; and
the CYP3A4 expression level increased by 12.8, 9.7 and 5.3 times, respectively,
as compared with the respective marker expression levels in the human induced hepatic stem cells AFB1-1 (No. 664) being taken as 1.

As is evident from the above-noted results, the culture procedures in the presence of dexamethasone (DEX) were suitable for effectively differentiating human induced hepatic progenitor cells from human induced hepatic stem cells. These culture procedures were also considered to be suitable for differentiating human hepatocytes from human induced hepatic progenitor cells.

TABLE 14
Culture conditions and results

| | Cell No. | | | | |
|---|---|---|---|---|---|
| | No. 663 | No. 664 | | | |
| | (Reference) | (Reference) | No. 686 | No. 687 | No. 688 |
| | Medium | | | | |
| | | | A-83-01 + | A-83-01 + | A-83-01 + |
| | | | 0.1 μM DEX/ | 0.5 μM DEX/ | 2.0 μM DEX/ |
| | ReproStem | mTeSR1 | ReproStem | ReproStem | ReproStem |
| bFGF | 10 | 100 | 0 | 0 | 0 |
| (ng/mL) | | | | | |
| Feeder | (+) | (+) | (−) | (−) | (−) |
| cells | | | | | |
| Matrigel | (+) | (+) | (+) | (+) | (+) |
| ALB | 1 | — | 3,706 | 4,306 | 2,559 |
| expression | | | | | |
| ratio | | | | | |
| AAT | 1 | — | 201 | 224 | 129 |
| expression | | | | | |
| ratio | | | | | |
| TTR | 1 | — | 156 | 166 | 89 |
| expression | | | | | |
| ratio | | | | | |
| AFP | 1 | — | 4,414 | 4,227 | 3,414 |
| expression | | | | | |
| ratio | | | | | |
| CYP1A2 | — | 1 | 6.4 | 4.9 | 10.8 |
| expression | | | | | |
| ratio | | | | | |
| CYP2C9 | — | 1 | 9.0 | 6.6 | 4.5 |
| expression | | | | | |
| ratio | | | | | |
| CYP3A4 | — | 1 | 12.8 | 9.7 | 5.3 |
| expression | | | | | |
| ratio | | | | | |

### Example 18

### Induction of Hepatic Differentiation, and Preparation of Induced Hepatic Progenitor Cells, in the Absence of bFGF/aFGF (2)

In a 10 cm-diameter culture dish coated with Matrigel (at an amount of 60 μL Matrigel/6 mL PBS/dish for about one hour), the human induced hepatic stem cells AFB1-1 (No. 663 (passage 35); about 50-80% confluence/dish), which were cocultured with feeder cells (about 1.5×10⁶ mouse embryonic fibroblasts (MEF)/60 cm² dish) using the human ES/iPS cell medium (ReproStem; ReproCELL) supplemented with 10 ng/mL bFGF and washed with PBS (−), were dissociated from the culture dish with a 0.25% trypsin/1 mM EDTA solution and suspended in the human ES/iPS cell medium (ReproStem; ReproCELL), then a tenth of the suspension was subjected to centrifugal washing (at 1,000 rpm for 5 minutes).

The human induced hepatic stem cells were suspended in the human ES/iPS cell medium (mTeSR1; STEMCELL Technologies)/Y-27632 (10 μM) and then seeded for coculture on feeder cells (about 1.5×10⁶ mouse embryonic fibroblasts (MEF)/60 cm² dish) seeded in a 10 cm-diameter culture dish coated with Matrigel (at an amount of 10 μL Matrigel/1 mL PBS/well for about one hour). The medium was replaced everyday with a fresh human ES/iPS cell medium (mTeSR1) to continue culture until reaching 50-80% confluence per dish.

The human induced hepatic stem cells AFB1-1 (No. 704 (passage 36)) were washed with PBS (−), and were then dissociated from the culture dish with a 0.25% trypsin/1 mM EDTA solution and suspended in the human ES/iPS cell medium (ReproStem; ReproCELL), then 1.2×10⁶ cells were subjected to centrifugal washing (at 1,000 rpm for 5 minutes). The human induced hepatic stem cells were suspended in the medium ReproStem (bFGF-free)/Y-27632 (10 μM) and then seeded (at a density of about 2×10⁵ cells/1 mL medium/well) in a 6-well plate coated with Matrigel (at an amount of 10 μL Matrigel/1 mL PBS/well for about one hour). After about three hours, the medium was replaced with 2 mL of ReproStem (bFGF-free) containing 0.5 μM A-83-01, and the human induced hepatic stem cells were subjected to culture for differentiation into human induced hepatic progenitor cells without feeder cells. The resultant cell sample is referred to as “No. 705”.

After the seeding, the medium was replaced every two or three days with a fresh medium of the same composition containing 0.1 μM A-83-01, and the cells were subjected to differentiation culture. Eight and thirteen days after the seeding, the culture supernatant was subjected to measurement (SRL Inc. (CRO)) for AFP, and 5,540 ng/mL and 2,320 ng/mL of AFPs were observed in No. 835 on the respective days. Thirteen days after the seeding, the cells were lysed in 1 mL/well of a QIAzol reagent to prepare the total RNA from the cell lysate using the miRNeasy Mini Kit (Qiagen). The total RNA was subjected to quantitative RT-PCR using the iScript Advanced cDNA synthesis kit, the SsoAdvanced SYBR Green Supremix (2 mL), and the CFX96 Real-Time System C1000 Thermal Cycler, all manufactured by Bio-Rad. The quantified genes were ALB, AAT, TTR, AFP, GATA4, SOX17, FOXA2, HNF4A, OCT3/4, NANOG, and SOX2.

According to the results of the quantitative RT-PCR, the expression levels of ALB, AAT, TTR, AFP, GATA4, SOX17, FOXA2 and HNF4A in the human induced hepatic progenitor cell sample (No. 705) increased by 51,653, 310, 2,282, 30,649, 1.44, 32.93, 1.19 and 5.42 times, respectively, and the expression levels of OCT3/4, NANOG and SOX2 in No. 705 decreased to 0.06, 0.01, and 0.01, respectively, as compared with the respective marker expression levels in the human induced hepatic stem cells (No. 663) being taken as 1.

As is evident from the above-noted results, the culture procedures in the presence of A-83-01 were suitable for effectively differentiating human induced hepatic progenitor cells from human induced hepatic stem cells. These culture procedures were also considered to be suitable for differentiating human hepatocytes from human induced hepatic stem cells or human induced hepatic progenitor cells. Further, the human induced hepatic stem cells expressed ALB, AAT, TTR, AFP, GATA4, SOX17, FOXA2, HNF4A, OCT3/4, NANOG and SOX2. The human induced hepatic progenitor cells increased in the expression levels of ALB, AAT, TTR and AFP; expressed GATA4, SOX17, FOXA2 and HNF4A; and decreased in the expression levels of OCT3/4, NANOG and SOX2.

TABLE 15
Culture conditions and results

| | Cell No. | | | |
|---|---|---|---|---|
| | No. 663 | | | |
| | (Reference) | No. 704 | No.705 | |
| | Medium | | | |
| | | | A-83-01/ReproStem | |
| | | | 8 days after | 13 days after |
| | ReproStem | mTeSR1 | the seeding | the seeding |
| bFGF | 10 | 100 | 0 | 0 |
| (ng/mL) | | | | |
| Feeder | (+) | (+) | (−) | (−) |
| cells | | | | |
| Matrigel | (+) | (+) | (+) | (+) |
| AFP | — | — | 5,540 ng/mL | 2,320 ng/mL |
| expression | | | | |
| level | | | | |
| ALB | 1 | — | — | 51,653 |
| expression | | | | |
| ratio | | | | |
| AAT | 1 | — | — | 310 |
| expression | | | | |
| ratio | | | | |
| TTR | 1 | — | — | 2,282 |
| expression | | | | |
| ratio | | | | |
| AFP | 1 | — | — | 30,649 |
| expression | | | | |
| ratio | | | | |
| GATA4 | 1 | — | — | 1.44 |
| expression | | | | |
| ratio | | | | |
| SOX17 | 1 | — | — | 32.93 |
| expression | | | | |
| ratio | | | | |
| FOXA2 | 1 | — | — | 1.19 |
| expression | | | | |
| ratio | | | | |
| HNF4A | 1 | — | — | 5.42 |
| expression | | | | |
| ratio | | | | |
| OCT3/4 | 1 | — | — | 0.06 |
| expression | | | | |
| ratio | | | | |
| NANOG | 1 | — | — | 0.01 |
| expression | | | | |
| ratio | | | | |
| SOX2 | 1 | — | — | 0.01 |
| expression | | | | |
| ratio | | | | |

### Example 19

### Induction of Hepatic Differentiation, and Preparation of Induced Hepatic Progenitor Cells, on Collagen Coat

In a 10 cm-diameter culture dish coated with Matrigel (at an amount of 60 μL Matrigel/6 mL PBS/dish for about one hour), the human induced hepatic stem cells AFB1-1, which were cocultured with feeder cells (about 1.5×10⁶ mouse embryonic fibroblasts (MEF)/60 cm² dish) using the human ES/iPS cell medium (ReproStem; ReproCELL) supplemented with 10 ng/mL bFGF and washed with PBS (−), were dissociated from the culture dish with a 0.25% trypsin/1 mM EDTA solution and suspended in the human ES/iPS cell medium (ReproStem; ReproCELL), then a tenth of the suspension was subjected to centrifugal washing (at 1,000 rpm for 5 minutes).

The human induced hepatic stem cells were suspended in the human ES/iPS cell medium (mTeSR1; STEMCELL Technologies)/Y-27632 (10 μM) and then seeded for coculture on feeder cells (about 1.5×10⁶ mouse embryonic fibroblasts (MEF)/60 cm² dish) seeded in a 10 cm-diameter culture dish coated with Matrigel (at an amount of 10 μL Matrigel/1 mL PBS/well for about one hour). The medium was replaced everyday with a fresh human ES/iPS cell medium (mTeSR1) to continue culture until reaching 50-80% confluence per dish. The human induced hepatic stem cells AFB1-1 (No. 631 (passage 34)) were washed with PBS (−), and were then dissociated from the culture dish with a 0.25% trypsin/1 mM EDTA solution and suspended in the human ES/iPS cell medium (ReproStem; ReproCELL), then 2.4×10⁶ cells were subjected to centrifugal washing (at 1,000 rpm for 5 minutes). The human induced hepatic stem cells were suspended in the medium ReproStem (bFGF-free)/Y-27632 (10 μM) and then seeded (at a density of about 4×10⁵ cells/1 mL medium/well) in the IWAKI 6-well collagen plate (No. 634) or the 6-well collagen plate coated with Matrigel (at an amount of 10 μL Matrigel/1 mL PBS/well for about one hour) (No. 637). After about three hours, the medium was replaced with 2 mL of ReproStem (bFGF-free) containing 0.1 μM A-83-01, and the human induced hepatic stem cells were subjected to culture for differentiation into human induced hepatic progenitor cells without feeder cells. The resultant cell samples are respectively referred to as “No. 634” and “No. 637”.

After the seeding, the medium was replaced every two or three days with a fresh medium of the same composition containing 0.1 μM A-83-01, and the cells were subjected to differentiation culture. Six and fourteen days after the seeding, the culture supernatant was subjected to measurement (SRL Inc. (CRO)) for AFP. As a result, 2,890 ng/mL and 3,040 ng/mL of AFPs were observed in Nos. 634 and 637, respectively, six days after the seeding, and 24,900 ng/mL and 30,000 ng/mL in respective samples fourteen days after the seeding. Also, fourteen days after the seeding, the cells were lysed in 1 mL/well of a QIAzol reagent to prepare the total RNA from the cell lysate using the miRNeasy Mini Kit (Qiagen). The total RNA was subjected to quantitative RT-PCR using the iScript Advanced cDNA synthesis kit, the SsoAdvanced SYBR Green Supremix (2 mL), and the CFX96 Real-Time System C1000 Thermal Cycler, all manufactured by Bio-Rad. The quantified genes were ALB, AAT, TTR and AFP.

According to the results of the quantitative RT-PCR, the expression level of ALB in the human induced hepatic progenitor cells (Nos. 634 and 637) increased by 246,304 and 244,450 times, respectively, as compared with the marker expression level in the human induced hepatic stem cells (No. 631) being taken as 1, which were cultured using the human ES/iPS cell medium (ReproStem) supplemented with 10 ng/mL bFGF. Also in Nos. 634 and 637, the AAT expression level increased by 236.13 and 236.51 times, respectively, the TTR expression level by 9,499 and 8,350 times, respectively, and the AFP expression level by 5,066 and 6,011 times, respectively.

As is evident from the above-noted results, the culture procedure on a collagen coat or a collagen/Matrigel coat was suitable for effectively differentiating human induced hepatic progenitor cells from human induced hepatic stem cells. This culture procedure was also considered to be suitable for differentiating human hepatocytes from human induced hepatic stem cells or human induced hepatic progenitor cells.

TABLE 16
Culture conditions and results

| | Cell No. | | | | |
|---|---|---|---|---|---|
| | No. 631 | | | | |
| | (Reference) | No. 634 | | No. 637 | |
| | Medium | | | | |
| | | A-83-01/ReproStem | | A-83-01/ReproStem | |
| | | 6 days after | 14 days after | 6 days after | 14 days after |
| | ReproStem | the seeding | the seeding | the seeding | the seeding |
| bFGF | 10 | 0 | 0 | 0 | 0 |
| (ng/mL) | | | | | |
| Feeder | (+) | (−) | (−) | (−) | (−) |
| cells | | | | | |
| Matrigel | (+) | (−) + | (−) + | (+) + | (+) + |
| | | collagen | collagen | collagen | collagen |
| AFP | — | 2,890 ng/mL | 24,900 ng/mL | 3,040 ng/mL | 30,000 ng/mL |
| expression | | | | | |
| level | | | | | |
| ALB | 1 | — | 246,304 | — | 244,450 |
| expression | | | | | |
| ratio | | | | | |
| AAT | 1 | — | 236.13 | — | 236.51 |
| expression | | | | | |
| ratio | | | | | |
| TTR | 1 | — | 9,499 | — | 8,350 |
| expression | | | | | |
| ratio | | | | | |
| AFP | 1 | — | 5,066 | — | 6,011 |
| expression | | | | | |
| ratio | | | | | |

1. A method of differentiating an induced hepatic stem cell into an induced hepatic progenitor cell or a hepatocyte, which comprises the step of culturing the induced hepatic stem cell for 1 to 4 weeks in the presence of a TGF-β inhibitor.

2. A method of differentiating an induced hepatic progenitor cell into a hepatocyte, which comprises the step of culturing the induced hepatic progenitor cell for 1 to 4 weeks in the presence of a TGF-β inhibitor.

3. The method cell according to claim 1 or 2, wherein the TGF-β inhibitor is selected from the group consisting of:
A-83-01 (3-(6-methylpyridin-2-yl)-1-phenylthiocarbamoyl-4-quinolin-4-ylpyrazole);
ALK5 Inhibitor I (3-pyridin-2-yl)-4-(4-quinonyl)-1H-pyrazole);
LDN193189 (4-(6-(4-(piperazin-1-yl)phenyl)pyrazolo[1,5-a]pyrimidin-3-yl)quinoline);
SB431542 (4-[4-(1,3-benzodioxol-5-yl)-5-pyridin-2-yl-1H-imidazol-2-yl]benzamide);
SB-505124 (2-(5-benzo[1,3]dioxol-5-yl-2-tert-butyl-3H-imidazol-4-yl)-6-methylpyridine hydrochloride hydrate);
SD-208 ((2-(5-chloro-2-fluorophenyl)pteridin-4-yl)pyridin-4-yl-amine);
SB-525334 (6-[2-(1,1-dimethylethyl)-5-(6-methyl-2-pyridinyl)-1H-imidazol-4-yl]quinoxaline);
LY-364947 (4-[3-(2-pyridinyl)-1H-pyrazol-4-yl]-quinoline);
LY2157299 (4-[2-(6-methyl-pyridin-2-yl)-5,6-dihydro-4H-pyrrolo[1,2-b]pyrazol-3-yl]-quinoline-6-carboxylic acid amide);
TGF-β RI Kinase Inhibitor II 616452 (2-(3-(6-methylpyridin-2-yl)-1H-pyrazol-4-yl)-1,5-naphthyridine);
TGF-β RI Kinase Inhibitor III 616453 (2-(5-benzo[1,3]dioxol-4-yl-2-tert-butyl-1H-imidazol-4-yl)-6-methylpyridine, HCl);
TGF-β RI Kinase Inhibitor IX 616463 (4-((4-((2,6-dimethylpyridin-3-yl)oxy)pyridin-2-yl)amino)benzenesulfonamide);
TGF-β RI Kinase Inhibitor VII 616458 (1-(2-((6,7-dimethoxy-4-quinolyl)oxy)-4,5-dimethylphenyl)-1-ethanone);
TGF-β RI Kinase Inhibitor VIII 616459 (6-(2-tert-butyl-5-(6-methyl-pyridin-2-yl)-1H-imidazol-4-yl)-quinoxaline);
AP12009 (TGF-β2 antisense compound “Trabedersen”);
Belagenpumatucel-L (TGF-β2 antisense gene modified allogenic tumor cell vaccine);
CAT-152 (Glaucoma-lerdelimumab (anti-TGF-β-2 monoclonal antibody));
CAT-192 (Metelimumab (human IgG4 monoclonal antibody which neutralizes TGFβ1));
GC-1008 (anti-TGF-β monoclonal antibody).

4. The method according to claim 1, wherein the culture is performed in the absence of bFGF.

5. The method according to claim 1, wherein the culture is performed in the absence of a feeder cell.

6. The method according to claim 1, wherein the culture is performed in the presence of a substance selected from the group consisting of matrigel and collagen.

7. The method according to claim 1, wherein the induced hepatic stem cell is subjected to preliminary culture in a pluripotent stem cell culture medium in the presence of a feeder cell followed by performing further culture in the presence of the TGF-β inhibitor.

8. The method according to claim 1, wherein the culture is performed in the presence of a substance selected from among a compound having a steroid skeleton, a fatty acid, and serum.

9. An induced hepatic progenitor cell which is characterized by satisfying at least the following two requirements (1) and (2):
(1) it expresses the OCT3/4, SOX2 and NANOG genes which are marker genes for an embryonic stem cell; and
(2) it expresses DLK1 and AFP which are hepatic stem/progenitor cell markers, as well as ALB, AAT and TTR which are hepatocyte markers.

10. The induced hepatic progenitor cell according to claim 9, wherein requirement (2) is that said induced hepatic progenitor cell expresses the hepatic stem/progenitor cell markers DLK1 and AFP, as well as the hepatocyte markers ALB, AAT, TTR, FGG, AHSG, FABP1, RBP4, TF and APOA4.

11. The induced hepatic progenitor cell according to claim 9 or 10, in which the OCT3/4, SOX2, and NANOG genes as the marker genes for an embryonic stem cell in the requirement (1) are expressed in amounts 1/10- 1/100 times as compared to the amounts of said genes as expressed in the embryonic stem cell or induced hepatic stem cell.

12. The induced hepatic progenitor cell according to claim 9, in which the DLK1 and AFP genes as the hepatic stem/progenitor cell markers in the requirement (2) are expressed in amounts 10 to 50,000 times as compared to the amounts of said genes as expressed in the embryonic stem cell or induced hepatic stem cell.

13. The induced hepatic progenitor cell according to claim 9, in which ALB, AAT, TTR, FGG, AHSG, FABP1, RBP4, TF and APOA4 genes as the hepatocyte markers in the requirement (2) are expressed in amounts 10 to 50,000 times as compared to the amounts of said genes as expressed in the embryonic stem cell or induced hepatic stem cell.

14. The induced hepatic progenitor cell according to claim 9, which is capable of adhesion culture or suspension culture for 1 to 2 weeks.

15. The induced hepatic progenitor cell according to claim 9, which further expresses the biliary duct epithelial cell marker KRT7.

16. The induced hepatic progenitor cell according to claim 9, which further expresses the hepatocyte growth factor HGF.

17. The induced hepatic progenitor cell according to claim 9, which is prepared by differentiating an induced hepatic stem cell through culture for 1 to 4 weeks in the presence of a TGF-β inhibitor.

18. A process for producing induced hepatic progenitor cells or hepatocytes, which comprises the step of performing the method according to claim 1.
